(19) United States
(12) Patent Application Publication
(10) Pub. No.: US 2003/0157637 A1
(43) Pub. Date: Aug. 21, 2003

(54) Diagnostics and treatments of periodontal disease

(75) Inventors: Eric Charles Reynolds, Victoria (AU); Peter Singh Bhogal, Victoria (AU); Nada Slakeski, Victoria (AU)

Correspondence Address: NIXON & VANDERHYE P.C., 1100 North Glebe Road, 8th Floor, Arlington, VA, 22201-4714, US

(73) Assignee: The University of Melbourne

(21) Appl. No.: 10/229,066

(22) Filed: Aug. 28, 2002

Related U.S. Application Data
(63) Continuation of application No. 09/066,330, filed on Sep. 15, 1998.

(51) Int. Cl. C12P 21/06

(52) U.S. Cl. 435/69.1

(57) ABSTRACT

This invention relates to the PrtR-PrtK cell surface protein of Porphyromonas gingivalis and in particular a multimeric cell associated protein complex comprising the PrtR and PrtK proteins. Accordingly the invention provides a substantially purified antigenic complex for use in raising an antibody response directed against Porphyromonas gingivalis. The complex comprises at least one multimeric protein complex of arginine-specific and lysine-specific thiol endopeptidases each containing at least one adhesin domain, the complex having a molecular weight of greater than about 200 kDa. The invention also relates to pharmaceutical compositions and associated agents based on said complex for the detection, prevention and treatment of Periodontal disease associated with P. gingivalis.

### FIELD OF THE INVENTION

This invention relates to the PrtR-PrtK cell surface protein of Porphyromonas gingivalis and in particular a multimeric cell associated protein complex comprising the PrtR and PrtK proteins. The invention also relates to pharmaceutical compositions and associated agents based on said complex for the detection, prevention and treatment of Periodontal disease associated with P. gingivalis.

### BACKGROUND OF THE INVENTION

Periodontal diseases are bacterial-associated inflammatory diseases of the supporting tissues of the teeth and range from the relatively mild form of gingivitis, the non-specific, reversible inflammation of gingival tissue to the more aggressive forms of periodontitis which are characterised by the destruction of the tooth's supporting structures. Periodontitis is associated with a subgingival infection of a consortium of specific Gram-negative bacteria that leads to the destruction of the periodontium and is a major public health problem. One bacterium that has attracted considerable interest is P. gingivalis as the recovery of this microorganism from adult periodontitis lesions can be up to 50% of the subgingival anaerobically cultivable flora, whereas P. gingivalis is rarely recovered, and then in low numbers, from healthy sites. A proportional increase in the level of P. gingivalis in subgingival plaque has been associated with an increased severity of periodontitis and eradication of the microorganism from the cultivable subgingival microbial population is accompanied by resolution of the disease. The progression of periodontitis lesions in non-human primates has been demonstrated with the subgingival implantation of P. gingivalis. These findings in both animals and humans suggest a major role for P. gingivalis in the development of adult periodontitis.

P. gingivalis is a black-pigmented, anaerobic, asaccharolytic, proteolytic Gram-negative rod that obtains energy from the metabolism of specific amino acids. The microorganism has an absolute growth requirement for iron, preferentially in the form of haeme or its Fe(III) oxidation product haemin and when grown under conditions of excess haemin is highly virulent in experimental animals. A number of virulence factors have been implicated in the pathogenicity of P. gingivalis including the capsule, adhesins, cytotoxins and extracellular hydrolytic enzymes. In particular, proteases have received a great deal of attention for their ability to degrade a broad range of host proteins including structural proteins and others involved in defence. The proteins that have been shown to be substrates for P. gingivalis proteolytic activity include collagen types I and IV, fibronectin, fibrinogen, laminin, complement and plasma clotting cascade proteins, α₁-antitrypsin, α₂-macroglobulin antichymotrypsin, antithrombin III, antiplasmin, cystatin C, IgG and IgA. The major proteolytic activities associated with this organism have been defined by substrate specificity and are “trypsin-like”, that is cleavage on the carboxyl side of arginyl and lysyl residues and collagenolytic although other minor activities have been reported.

P. gingivalis trypsin-like proteolytic activity has been shown to degrade complement, generating biologically active C5a, impair the phagocytic and other functions of neutrophils by modifying surface receptors, and abrogate the clotting potential of fibrinogen prolonging plasma clotting time. The trypsin-like proteolytic activity of P. gingivalis also generates Fc fragments from human IgG1 stimulating the release of pro-inflammatory cytokines from mononuclear cells and is associated with vascular disruption and enhanced vascular permeation through the activation of the kallikrein-kinin cascade. P. gingivalis spontaneous mutants with reduced trypsin-like activity as well as wild-type cells treated with the trypsin-like protease inhibitor N-p-tosyl-L-lysine chloromethyl ketone are avirulent in animal models. Further, it has been shown that P. gingivalis grown under controlled, haemin-excess conditions expressed more trypsin-like and less collagenolytic activity and were more virulent in mice relative to cells grown under haemin-limited but otherwise identical conditions. The increased expression of the trypsin-like activity by the more virulent P. gingivalis has led to the speculation that the trypsin-like proteolytic activity may be the major determinant for infection or disease. However, the cell-associated trypsin-like proteolytic activities of P. gingivalis have not been characterised to date.

There has been considerable endeavour to purify and characterise the trypsin-like proteases of P. gingivalis from cell-free culture fluids. Chen et al, (1992) [J Biol Chem 267:18896-18901] have purified and characterised a 50 kDa arginine-specific, thiol protease from the culture fluid of P. gingivalis H66 designated Arg-gingipain. A similar arginine-specific thiol protease has been disclosed in JP 07135973 and the amino acid sequence disclosed in WO 9507286 and in Kirszbaum et al, 1995 [Biochem Biophys Res Comm 207:424-431]. Pike et al (1994) [J Biol Chem 269:406-411] have characterised a 60 kDa lysine-specific cysteine proteinase from the culture fluid of P. gingivalis H66 designated Lys-gingipain and the partial gene sequence for this enzyme was disclosed in WO 9511298 and fully disclosed in WO 9617936. However, prior to the development of the present invention it was unknown that there existed on the cell surface of P. gingivalis a 300 kDa complex of arginine-specific and lysine-specific proteases both containing adhesin domains. The 300 kDa complex has been designated the PrtR-PrtK complex. The presence of the PrtR-PrtK cell surface complex exhibiting both arginine- and lysine-specific proteolytic activity together with adhesin activity was previously unknown. Furthermore, the new PrtR-PrtK complex of the present invention is expressed on the cell surface, is a major virulence-associated factor and contains unique epitopes not displayed on the individual domains. The previously disclosed arginine-specific and lysine-specific thiol proteases, as discussed, do not exhibit any of these features and have proven of limited application to date. However, the aforementioned features have rendered the PrtR-PrtK complex of the invention ideal for development of diagnostic and immunoprophylactic products. The PrtR-PrtK cell surface complex is accordingly of particular interest for diagnostics and neutralisation by passive immunity through oral compositions containing neutralising antibodies and by vaccine development. In particular for the development of an intra-oral recombinant bacterial vaccine, where the recombinant bacterium expressing an inactivated PrtR-PrtK is a genetically engineered commensal inhabitant of the oral cavity.

### SUMMARY OF THE INVENTION

Accordingly in a first aspect the present invention consists in a substantially purified antigenic complex for use in raising an antibody response directed against Porphyromonas gingivalis, the complex comprising at least one multimeric protein complex of arginine-specific and lysine-specific thiol endopeptidases each containing at least one adhesin domain, the complex having a molecular weight of greater than about 200 kDa.

In the context of this disclosure, the terms “adhesin” and “hemagglutinin” may be considered to be synonymous.

In a preferred form of the present invention the multimeric protein complex is associated with virulent strains of Porphyromonas gingivalis, preferably has a molecular weight of about 294 to about 323 kDa and is preferably derived from P. gingivalis W50.

It is also preferred that the multimeric protein complex is composed of 9 proteins. These 9 proteins preferably have the following N-terminal sequences:

DVYTDHGDLYNTPVRML
YTPVEEKQNGRMIVIVAKKYEGD
SGQAEIVLEAHDVWNDGSGYQILLDADHDQYGQVIPSDTHFL
PQSVWIERTVDLPAGTKYVAFR
ANEAKVVLAADNVWGDNTGYQFLLDA
ANEAKVVLAADNVWGDNTGYQFLLDA
PQSVWIERTVDLPAGTKYVAFR
ADFTETFESSTHGEAPAEWTTIDA
ADFTETFESSTHGEAPAEWTTIDA

It is presently preferred that the 9 proteins are PrtK49, PrtR45, PrtR44, PrtK39, PrtK44, PrtR27, PrtR17, PrtK15 and PrtR15 as described herein.

As the purified antigenic complex normally has enzymatic activity it is preferred in a number of uses the thiol endopeptidases are rendered inactive. This may be achieved in a number of ways, for example by oxidation or by mutation. It is presently preferred that the inactivation is by oxidation.

In yet another preferred embodiment of the present invention the multimeric protein complex is encoded by the DNA sequence shown in FIGS. 8B and 9B.

In a second aspect the present invention consists in a composition for use in eliciting an immune response directed against Porphyromonas gingivalis, the composition comprising an effective amount of the complex of the first aspect of the present invention and a suitable adjuvant and/or acceptable carrier.

In a third aspect the present invention consists in an antibody preparation comprising antibodies specifically directed against the complex of the first aspect of the present invention. The antibodies may be polyclonal antibodies or monoclonal antibodies.

In a fourth aspect the present invention consists in a method of treating a subject suffering from Porphyromonas gingivalis infection, the method comprising administering to the subject an amount of the antibody preparation of the third aspect of the present invention effective to at least partially neutralize the PrtR-PrtK complex of Porphyromonas gingivalis.

As will be recognised by those skilled in the art the antibody preparation may be administered by any of a number of well known routes, however, it is presently preferred that the preparation is administered orally.

In a fifth aspect the present invention consists in a method of reducing the prospect of P. gingivalis infection in an individual and/or severity of disease, the method comprising administering to the individual an amount of the composition of the second aspect of the present invention effective to induce an immune response in the individual directed against P. gingivalis.

In yet a further aspect the present invention consists in a recombinant host cell, the host cell being transformed with a DNA sequence(s) encoding PrtR-PrtK operatively linked to control sequences such that under appropriate conditions the host cell expresses PrtR-PrtK.

In another aspect, the present invention is directed to novel DNA sequences involving PrtR-PrtK constructs and vectors including plasmid DNA, and viral DNA such as human viruses, animal viruses, insect viruses, or bacteriophages which can be used to direct the expression of PrtR-PrtK protein in appropriate host cells from which the expressed protein may be purified. Another aspect of the present invention provides methods for molecular cloning of the genes encoding the PrtR-PrtK complex. The nucleic acid sequences of the present invention can be used in molecular diagnostic assays for P. gingivalis genetic material through nucleic acid hybridization, and including the synthesis of PrtR-PrtK sequence-specific olizonucleotides for use as primers and/or probes in amplifying, and detecting amplified, nucleic acids. Additionally, PrtR-PrtK complex can be used as an immunogen in prophylactic and/or therapeutic vaccine formulations against pathogenic strains of P. gingivalis, whether the immunogen is chemically synthesized, purified from P. gingivalis, or purified from a recombinant expression vector system. Alternatively, the genes encoding PrtR-PrtK may be incorporated into a bacterial or viral vaccine comprising recombinant bacteria or virus which is engineered to produce PrtR-PrtK by itself, or in combination with immunogenic epitopes of other pathogenic microorganisms. In addition, the genes encoding PrtR-PrtK operatively linked to one or more regulatory elements, can be introduced directly into humans to express the PrtR-PrtK to elicit a protective immune response. A vaccine can also be based upon a recombinant component of a mutated PrtR-PrtK incorporated into an appropriate vector and expressed in a suitable transformed host (eg. E. coli, Bacillus subtilis, Saccharomyces cerevisiae, COS cells, CHO cells and HeLa cells) containing the vector. The vaccine can be based on an intra-oral recombinant bacterial vaccine, where the recombinant bacterium expressing an inactivated PrtR-PrtK is a commensal inhabitant of the oral cavity. Unlike whole P. gingivalis cells or other previously prepared antigens based on fimbriae or the capsule the PrtR-PrtK complex of the invention or component parts thereof are safe and effective antigens for the preparation of a vaccine for the prevention of P. gingivalis-associated periodontal disease. The invention therefore provides a range of recombinant products based on the PrtR-PrtK complex.

The invention also provides antibodies raised against the said PrtR-PrtK complex, herein called anti-PrtR-PrtK antibodies. The antibodies may be blended into oral compositions such as toothpaste, mouthwash, toothpowders and liquid dentifrices, mouthwashes, troches, chewing gums, dental pastes, gingival massage creams, gargle tablets, dairy products and other foodstuffs

In another aspect the invention provides a method of diagnosis for the presence of P. gingivalis characterised by the use of any one or a combination of an antibody, antigen or nucleic acid probe as hereinbefore defined comprising the application of known techniques including for example, enzyme linked immunosorbent assay.

The invention also provides diagnostic kits comprising antibodies, antigens and/or nucleic acid probes as hereinbefore defined.

### BRIEF DESCRIPTION OF FIGURES

FIG. 1. Anion exchange FPLC of a P. gingivalis W50 sonicate. The sonicate in TC buffer containing 50 mM NaCl was applied to a Hiload XK 16/10 Q sepharose column and eluted using a linear gradient from 0 - 100% buffer B over 90 min at a flow rate of 2.0 ml min⁻¹ Fractions (6 ml) were assayed for proteolytic and amidolytic activity using azocasein, Bz-L-Arg-pNA and Z-L-Lys-pNA. The amidolytic activity of each 6 ml fraction with Bz-L-Arg-pNA is shown by the histogram.

FIG. 2. Gel filtration FPLC of the pooled and concentrated fractions from Q sepharose anion exchange FPLC containing proteolytic/amidolytic activity. Anion exchange fractions containing the major peak of proteolytic/amidolytic activity were pooled, equilibrated in TC buffer containing 150 mM NaCl, concentrated and divided into four aliquots and each then independently applied to a gel filtration column (Superose 12 HR 10/30) and eluted using the same buffer at a flow rate of 0.3 ml min⁻¹. Fractions (0.5 ml) were assayed for proteolytic and amidolytic activity. Bz-L-Arg-pNA amidolytic activity is shown by the histogram. Vo and Vt indicate the void and total volumes of the column respectively. The elution volumes of the standard proteins thyroglobulin 667 kDa, catalase 232 kDa and aldolase 158 kDa are marked.

FIG. 3. SDS-PAGE (boiled/reduced conditions) of the 300 kDa peak from gel filtration (Superose 12 HR 10/30) FPLC. Lane 1, Pharmacia molecular mass standards (Mᵣ shown in kDa). Lane 2, 300 kDa peak from gel filtration FPLC. Coomassie blue stained gel.

FIG. 4. Specific cleavage sites (marked with arrows) of αₛ₁-casein by the proteolytic/amidolytic peak from gel filtration FPLC corresponding to 300 kDa. The protein αₛ₁-casein was cleaved on the carboxyl side of arginyl and lysyl residues only.

FIG. 5. Arg-sepharose FPLC of the 300 kDa gel filtration peak exhibiting Arg- and Lys-specific proteolytic activity. Gel filtration fractions containing the major peak of proteolytic activity (300 kDa) were pooled and applied to an arginine-sepharose column (5 ml arginine-Sepharose 4B) and washed with TC buffer containing 50 mM NaCl at 0.1 ml min⁻¹ until the baseline returned to zero. The column was then further washed with 500 mM NaCl and then re-equilibrated with TC buffer containing 50 mM NaCl. The column was first eluted with 200 mM lysine in TC buffer containing 50 mM NaCl, followed by 750 mM lysine in the same buffer. The column was then re-equilibrated and eluted with 200 mM arginine in the same buffer at a flow rate of 0.1 ml min⁻¹. Peaks were collected and assayed for amidolytic and proteolytic activity Bz-L-Arg-pNA amidolytic activity is shown by the histogram and the arrows indicate the start of each step gradient.

FIG. 6. SDS-PAGE (boiled/reduced conditions) of 200 mM lysine eluant from the Arg-sepharose FPLC. Lane 1, Pharmacia molecular mass standards (Mᵣ shown in kDa) Lane 2, 200 mM lysine eluant from Arg-sepharose FPLC Silver stained gel.

FIG. 7. SDS-PAGE (boiled/reduced conditions) of the 750 mM lysine and 200 mM arginine eluants from the arginine-Sepharose FPLC and the purified 45 kDa Arg-specific endopeptidase. Lane 1, 750 mM lysine eluant. Lane 2, 200 mM arginine eluant. Lane 3, purified 45 kDa Arg-specific endopeptidase. Lane 4, Pharmacia molecular mass standards (Mᵣ shown in kDa). Coomassie blue stained gel.

FIG. 8a. Schematic representation of the prtR gene. The PrtR nascent polyprotein is composed of a leader sequence, a prosequence followed by the Arg-specific cysteine proteinase PrtR45, and the adhesins PrtR44, PrtR15, PrtR17 and PrtR27 all preceded by an arginyl or lysyl residue.

FIG. 8b. Nucleotide sequence of prtR.

FIG. 9a. Schematic representation of the prtK gene. The PrtK nascent polyprotein is composed of a leader sequence , a prosequence followed by the Lys-specific cysteine proteinase PrtK48, and the adhesins PrtK39, PrtK15 and PrtK44 all preceded by an arginyl or lysyl residue.

FIG. 9b. Nucleotide sequence of prtK.

FIG. 10. SDS-PAGE of the PrtR-PrtK complex purified by diafiltration. Lane 1 shows molecular mass markers. Lane 2 shows components of the PrtR-PrtK purified by diafiltration

FIG. 11. ELISA titration of sera from 5 mice immunized twice with the PrtR-PrtK complex emulsified in Freund's Incomplete Adjuvant. Test sera (TS 32-36) and pre-immune sera (PIS 32-36) were screened using P. gingivalis W50 sonicate as the adsorbed antigen. Primary antibody dilutions of 1/100, 1/500, 1/2500 and 1/12500 were used. Bound antibody was determined using horseradish peroxidase-conjugated goat anti-mouse antibody and 3,3′,5,5′ tetramethylbenezidine. The reaction product was quantitated spectrophotometrically using a 450 nm interference filter in a plate reader and recorded as optical density (O.D.) readings.

### DETAILED DESCRIPTION OF THE INVENTION

The invention will now be described in greater detail by reference to the methods used and applied in the development of the invention and by reference to particular examples which provide the best methods known of performing the invention.

The intra-oral bacterium Porphyromonas gingivalis possesses on its cell surface major trypsin-like proteinases as a 294-323 kDa heterodimeric protein complex of Arg-specific and Lys-specific thiol endopeptidases with hemagglutinins (adhesins) herein designated the PrtR-PrtK complex. The PrtR-PrtK complex can be purified from P. gingivalis cells by ultrasonication or chloroform extraction followed by diafiltration or anion exchange and Lys-sepharose or Arg-sepharose chromatography. The purified PrtR-PrtK complex is then used to generate antibodies using standard techniques. The animals used for antibody generation can be rabbits, goats, chickens, sheep, horses, cows etc. When a high antibody titre against the PrtR-PrtK complex is detected by immunoassay the animals are bled or eggs or milk are collected and the serum prepared and/or antibody purified using standard techniques or monoclonal antibodies produced by fusing spleen cells with myeloma cells using standard techniques. The antibody (immunoglobulin fraction) may be separated from the culture or ascites fluid, serum, milk or egg by salting out, gel filtration, ion exchange and/or affinity chromatography, and the like, with salting out being preferred. In the salting out method the antiserum or the milk is saturated with ammonium sulphate to produce a precipitate, followed by dialyzing the precipitate against physiological saline to obtain the purified immunoglobulin fraction with the specific anti-(PrtR-PrtK). The preferred antibody is obtained from the equine antiserum and the bovine antiserum and milk. In this invention the antibody contained in the antiserum and milk obtained by immunising the animal with the inactivated PrtR-PrtK may be blended into the oral composition. In this case the antiserum and milk as well as the antibody separated and purified from the antiserum and milk may be used. Each of these materials may be used alone or in combination of two or more. Antibodies against the PrtR-PrtK can be used in oral compositions such as toothpaste and mouthwash to neutralise the PrtR-PrtK and thus prevent disease. The anti-(PrtR-PrtK) antibodies can also be used for the early detection of P. gingivalis in subgingival plaque samples by a chairside Enzyme Linked Immunosorbent Assay (ELISA).

For oral compositions it is preferred that the amount of the above antibodies administered is 0.0001 -50 g/kg/day and that the content of the above antibodies is 0.0002 - 10% by weight preferably 0.002 -5% by weight of the composition. The oral composition of this invention which contains the above-mentioned serum or milk antibody may be prepared and used in various forms applicable to the mouth such as dentifrice including toothpastes, toothpowders and liquid dentifrices, mouthwashes, troches, periodontal pocket irrigating devices, chewing gums, dental pastes, gingival massage creams, gargle tablets, dairy products and other foodstuffs. The oral composition according to this invention may further include additional well known ingredients depending on the type and form of a particular oral composition.

In certain highly preferred forms of the invention the oral composition may be substantially liquid in character, such as a mouthwash or rinse. In such a preparation the vehicle is typically a water-alcohol mixture desirably including a humectant as described below. Generally, the weight ratio of water to alcohol is in the range of from about 1:1 to about 20:1. The total amount of water-alcohol mixture in this type of preparation is typically in the range of from about 70 to about 99.9% by weight of the preparation. The alcohol is typically ethanol or isopropanol. Ethanol is preferred.

The pH of such liquid and other preparations of the invention is generally in the range of from about 4.5 to about 9 and typically from about 5.5 to 8. The pH is preferably in the range of from about 6 to about 8.0, preferably 7.4. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc).

Other desirable forms of this invention, the oral composition may be substantially solid or pasty in character, such as toothpowder, a dental tablet or a dentifrice, that is a toothpaste (dental cream) or gel dentifrice. The vehicle of such solid or pasty oral preparations generally contains dentally acceptable polishing material. Examples of polishing materials are water-insoluble sodium metaphosphate, potassium metaphosphate, tricalcium phosphate, dihydrated calcium phosphate, anhydrous dicalcium phosphate, calcium pyrophosphate, magnesium orthophosphate, trimagnesium phosphate, calcium carbonate, hydrated alumina, calcined alumina, aluminium silicate, zirconium silicate, silica, bentonite, and mixtures thereof. Other suitable polishing material include the particulate thermosetting resins such as melamine-, phenolic, and urea-formaldehydes, and cross-linked polyepoxides and polyesters. Preferred polishing materials include crystalline silica having particle sized of up to about 5 microns, a mean particle size of up to about 1.1 microns, and a surface area of up to about 50,000 cm²/gm., silica gel or colloidal silica, and complex amorphous alkali metal aluminosilicate.

When visually clear gels are employed, a polishing agent of colloidal silica, such as those sold under the trademark SYLOID as Syloid 72 and Syloid 74 or under the trademark SANTOCEL as Santocel 100, alkali metal alumino-silicate complexes are particularly useful since they have refractive indices close to the refractive indices of gelling agent-liquid (including water and/or humectant) systems commonly used in dentifrices.

Many of the so-called “water insoluble” polishing materials are anionic in character and also include small amounts of soluble material. Thus, insoluble sodium metaphosphate may be formed in any suitable manner as illustrated by Thorpe's Dictionary of Applied Chemistry, Volume 9, 4th Edition, pp. 510-511. The forms of insoluble sodium metaphosphate known as Madrell's salt and Kurrol's salt are further examples of suitable materials. These metaphosphate salts exhibit only a minute solubility in water, and therefore are commonly referred to as insoluble metaphosphates (IMP). There is present therein a minor amount of soluble phosphate material as impurities, usually a few percent such as up to 4% by weight. The amount of soluble phosphate material, which is believed to include a soluble sodium trimetaphosphate in the case of insoluble metaphosphate, may be reduced or eliminated by washing with water if desired. The insoluble alkali metal metaphosphate is typically employed in powder form of a particle size such that no more than 1% of the material is larger than 37 microns.

The polishing material is generally present in the solid or pasty compositions in weight concentrations of about 10% to about 99%. Preferably, it is present in amounts from about 10% to about 75% in toothpaste, and from about 70% to about 99% in toothpowder. In toothpastes, when the polishing material is silicious in nature, it is generally present in amount of about 10-30% by weight. Other polishing materials are typically present in amount of about 30-75% by weight.

In a toothpaste, the liquid vehicle may comprise water and humectant typically in an amount ranging from about 10% to about 80% by weight of the preparation. Glycerine, propylene glycol, sorbitol and polypropylene glycol exemplify suitable humectants/carriers Also advantageous are liquid mixtures of water, glycerine and sorbitol. In clear gels where the refractive index is an important consideration, about 2.5 - 30% w/w of water, 0 to about 70% w/w of glycerine and about 20-80% w/w of sorbitol are preferably employed.

Toothpaste, creams and gels typically contain a natural or synthetic thickener or gelling agent in proportions of about 0.1 to about 10, preferably about 0.5 to about 5% w/w. A suitable thickener is synthetic hectorite, a synthetic colloidal magnesium alkali metal silicate complex clay available for example as Laponite (e.g. CP, SP 2002, D) marketed by Laporte Industries Limited. Laponite D is, approximately by weight 58.00% SiO₂, 25.40% MgO, 3.05% Na₂O, 0.98% Li₂O, and some water and trace metals. Its true specific gravity is 2.53 and it has an apparent bulk density of 1.0 g/ml at 8% moisture.

Other suitable thickeners include Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose (e.g. available as Natrosol), sodium carboxymethyl cellulose, and colloidal silica such as finely ground Syloid (e.g. 244). Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate.

It will be understood that, as is conventional, the oral preparations are to be sold or otherwise distributed in suitable labelled packages. Thus, a jar of mouthrinse will have a label describing it, in substance, as a mouthrinse or mouthwash and having directions for its use, and a toothpaste, cream-or gel will usually be in a collapsible tube, typically aluminium, lined lead or plastic, or other squeeze, pump or pressurised dispenser for metering out the contents, having a label describing it, in substance, as a toothpaste, gel or dental cream.

Organic surface-active agents are used in the compositions of the present invention to achieve increased prophylactic action, assist in achieving thorough and complete dispersion of the active agent throughout the oral cavity, and render the instant compositions more cosmetically acceptable. The organic surface-active material is preferably anionic, nonionic or ampholytic in nature which does not denature the antibody of the invention, and it is preferred to employ as the surface-active agent a detersive material which imparts to the composition detersive and foaming properties while not denaturing the antibody. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkylsulfo-acetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarconite compounds in the oral compositions of the present invention is particularly advantageous since these materials exhibit a prolonged marked effect in the inhibition of acid formation in the oral cavity due to carbohydrates breakdown in addition to exerting some reduction in the solubility of tooth enamel in acid solutions. Examples of water-soluble nonionic surfactants suitable for use with antibodies are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly (ethylene oxide) with fatty acids, fatty alcohols, fatty amides, polyhydric alcohols (e.g. sorbitan monostearate) and polypropyleneoxide (e.g. Pluronic materials).

Surface active agent is typically present in amount of about 0.1-5% by weight. It is noteworthy, that the surface active agent may assist in the dissolving of the antibody of the invention and thereby diminish the amount of solubilizing humectant needed.

Various other materials may be incorporated in the oral preparations of this invention such as whitening agents, preservatives, silicones, chlorophyll compounds and/or ammoniated material such as urea, diammonium phosphate, and mixtures thereof. These adjuvants, where present, are incorporated in the preparations in amounts which do not substantially adversely affect the properties and characteristics desired.

Any suitable flavouring or sweetening material may also be employed. Examples of suitable flavouring constituents are flavouring oils, e.g. oil of spearmint, peppermint, wintergreen, sassafras, clove, sage, eucalyptus, marjoram, cinnamon, lemon and orange, and methyl salicylate. Suitable sweetening agents include sucrose, lactose, maltose, sorbitol, xylitol, sodium cyclamate, perillartine, AMP (aspartyl phenyl alanine, methyl ester), saccharine, and the like. Suitably, flavour and sweetening agents may each or together comprise from about 0.1% to 5% more of the preparation.

In the preferred practice of this invention an oral composition according to this invention such as mouthwash or dentifrice containing the composition of the present invention is preferably applied regularly to the gums and teeth, such as every day or every second or third day or preferably from 1 to 3 times daily, at a pH of about 4.5 to about 9, generally about 5.5 to about 8, preferably about 6 to 8, for at least 2 weeks up to 8 weeks or more up to a lifetime.

The compositions of this invention can be incorporated in lozenges, or in chewing gum or other products, e.g. by stirring into a warm gum base or coating the outer surface of a gum base, illustrative of which may be mentioned jelutong, rubber latex, vinylite resins, etc., desirably with conventional plasticisers or softeners, sugar or other sweeteners or such as glucose, sorbitol and the like.

The composition of this invention also includes targeted delivery vehicles such as periodontal pocket irrigation devices, collagen, elastin, or synthetic sponges, membranes or fibres placed in the periodontal pocket or used as a barrier membrane or applied directly to the tooth root.

Another important form of the invention is a composition for use in eliciting an immune response directed against Porphyromonas gingivalis based on the PrtR-PrtK complex and suitable adjuvant delivered by nasal spray, orally or by injection to produce a specific immune response against the PrtR-PrtK complex thereby reducing colonisation of P. gingivalis and neutralising the PrtR-PrtK thereby preventing disease. Due to the potent enzymatic activity of the complex typically the complex will be inactivated. A vaccine can also be based upon a recombinant component of the PrtR-PrtK incorporated into an appropriate vector and expressed in a suitable transformed host (eg. E. coli, Bacillus subtilis, Saccharomyces cerevisiae, COS cells, CHO cells and HeLa cells) containing the vector Unlike whole P. gingivalis cells or other previously prepared antigens based on fimbriae or the capsule the PrtR-PrtK complex is a safe and effective antigens for the preparation of a composition for use in the prevention of P. gingivalis-associated periodontal disease. The PrtR-PrtK complex can be produced using recombinant DNA methods as illustrated herein, or can be synthesized chemically from the amino acid sequence disclosed in the present invention. Additionally, according to the present invention, the PrtR-PrtK complex may be used to generate antisera useful for passive immunization against periodontal disease and infections caused by P. gingivalis

Various adjuvants are used in conjunction with vaccine formulations. The adjuvants aid by modulating the immune response and in attaining a more durable and higher level of immunity using smaller amounts of vaccine antigen or fewer doses than if the vaccine antigen were administered alone. Examples of adjuvants include incomplete Freund's adjuvant (IFA), Adjuvant 65 (containing peanut oil, mannide monooleate and aluminium monostrearate), oil emulsions, Ribi adjuvant, the pluronic polyols, polyamines, Avridine, Quil A, saponin, MPL, QS-21, and mineral gels such as aluminium salts. Other examples include oil in water emulsions such as SAF-1, SAF-0, MF59, Seppic ISA720, and other particulate adjuvants such as ISCOMs and ISCOM matrix. An extensive but not exhaustive list of other examples of adjuvants are listed in Cox and Coulter 1992 [In: Wong WK (ed.) Animals parasite control utilising technology. Bocca Raton; CRC press, 1992; 49-112]. In addition to the adjuvant the vaccine may include conventional pharmaceutically acceptable carriers, excipients, fillers, buffers or diluents as appropriate. One or more doses of the vaccine containing adjuvant may be administered prophylactically to prevent periodontitis or therapeutically to treat already present periodontitis.

In another preferred composition the preparation is combined with a mucosal adjuvant and administered via the oral route. Examples of mucosal adjuvants are cholera toxin and heat labile E. coli toxin, the non-toxic B subunits of these toxins, genetic mutants of these toxins which have a reduced toxicity. Other methods which may be utilised to deliver the PrtR-PrtK complex orally include incorporation of the protease into particles of biodegradable polymers (such as acrylates or polyesters) by microencapsulation to aid uptake of the microspheres from the gastrointestinal tract and to protect degradation of the proteins. Liposomes, ISCOMs, hydrogels are examples of other potential methods which may be further enhanced by the incorporation of targeting molecules such as LTB, CTB or lectins for delivery of the PrtR-PrtK complex to the mucosal immune system. In addition to the vaccine and the mucosal adjuvant or delivery system the vaccine may include conventional pharmaceutically acceptable carriers, excipients, fillers, coatings, dispersion media, antibacterial and antifungal agents, buffers or diluents as appropriate.

Another mode of this embodiment provides for either a live recombinant viral vaccine, recombinant bacterial vaccine, recombinant attenuated bacterial vaccine, or an inactivated recombinant viral vaccine which is used to protect against infections caused by P. gingivalis. Vaccinia virus is the best known example, in the art, of an infectious virus that is engineered to express vaccine antigens derived from other organisms. The recombinant live vaccinia virus, which is attenuated or otherwise treated so that it does not cause disease by itself, is used to immunize the host. Subsequent replication of the recombinant virus within the host provides a continual stimulation of the immune system with the vaccine antigens such as PrtR-PrtK complex, thereby providing long lasting immunity.

Other live vaccine vectors include: adenovirus, cytomegalovirus, and preferably the poxviruses such as vaccinia (Paoletti and Panicali U.S. Pat. No. 4,603,112) and attenuated Salmonella strains (Stocker et al., U.S. Pat. Nos. 5,210,035; 4,837,151; and 4,735,801; and Curtiss et al., 1988, Vaccine 6:155-160). Live vaccines are particularly advantageous because they continually stimulate the immune system which can confer substantially long-lasting immunity. When the immune response is protective against subsequent P. gingivalis infection, the live vaccine itself may be used in a preventive vaccine against P. gingivalis. In particular, the live vaccine can be based on a bacterium that is a commensal inhabitant of the oral cavity. This bacterium can be transformed with a vector carrying a recombinant inactivated PrtR-PrtK and then used to colonise the oral cavity, in particular the oral mucosa. Once colonised the oral mucosa, the expression of the recombinant protein will stimulate the mucosal associated lymphoid tissue to produce neutralising antibodies. For example, using molecular biological techniques the genes encoding the PrtR-PrtK may be inserted into the vaccinia virus genomic DNA at a site which allows for expression of epitopes but does not negatively affect the growth or replication of the vaccinia virus vector The resultant recombinant virus can be used as the immunogen in a vaccine formulation. The same methods can be used to construct an inactivated recombinant viral vaccine formulation except that the recombinant virus is inactivated, such as by chemical means known in the art, prior to use as an immunogen and without substantially affecting the immunogenicity of the expressed immunogen.

In another variation of this embodiment, genetic material is used directly as the vaccine formulation. Nucleic acid (DNA or RNA) containing sequences encoding the PrtR-PrtK protein complex operatively linked to one or more regulatory elements can be introduced directly to vaccinate the individual (“direct gene transfer”) against pathogenic strains of P. gingivalis. Direct gene transfer into a vaccinated individual, resulting in expression of the genetic material by the vaccinated individual's cells such as vascular endothelial cells as well as the tissue of the major organs, has been demonstrated by techniques in the art such as by injecting intravenously an expression plasmid:cationic liposome complex (Zhu et al., 1993, Science 261.209-211). Other effective methods for delivering vector DNA into a target cell are known in the art. In one example, purified recombinant plasmid DNA containing viral genes has been used to inoculate (whether parentally, mucosally, or via gene-gun immunization) vaccines to induce a protective immune response (Fynan et al. 1993, Proc. Natl. Acad. Sci. USA 90:11478-11482). In another example, cells removed from an individual can be transfected or electroporated by standard procedures known in the art, resulting in the introduction of the recombinant vector DNA into the target cell. Cells containing the recombinant vector DNA may then be selected for using methods known in the art such as via a selection marker expressed in the vector, and the selected cells may then be re-introduced into the individual to express the PrtR-PrtK complex.

As an alternative to active immunization, immunization may be passive, i.e. immunization comprising administration of purified immunoglobulin containing antibody against PrtR-PrtK.

The present invention further provides the nucleotide sequence of the genes encoding the PrtR-PrtK complex, as well as the amino acid sequence deduced from the isolated genes. According to one embodiment of the present invention, using recombinant DNA techniques the genes encoding the PrtR-PrtK complex are incorporated into an expression vector, and the recombinant vector is introduced into an appropriate host cell thereby directing the expression of these sequences in that particular host cell. The expression system, comprising the recombinant vector introduced into the host cell, can be used (a) to produce PrtR-PrtK complex which can be purified for use as an immunogen in vaccine formulations, (b) to produce PrtR-PrtK complex to be used as an antigen for diagnostic immunoassays or for generating P. gingivalis-specific antisera of therapeutic and/or diagnostic value; (c) or if the recombinant expression vector is a live virus such as vaccinia virus, the vector itself may be used as a rive or inactivated vaccine preparation to be introduced into the host's cells for expression of PrtR-PrtK complex, (d) for introduction into live attenuated bacterial cells or genetically engineered commensal intra-oral bacteria which are used to express PrtR-PrtK complex to vaccinate individuals, (e) or for introduction directly into an individual to immunize against the encoded and expressed PrtR-PrtK complex. In particular the recombinant bacterial vaccine can be based on a commensal inhabitant of the human oral cavity or animal if the vaccine is to prevent periodontal disease in animals The recombinant bacterial vaccine expressing inactivated PrtR-PrtK can be used to colonise the oral cavity, supragingival or subgingival plaque. The intra-oral bacterium can be isolated from the patient with periodontitis and genetically engineered to express inactivated PrtR-PrtK complex. The production of the inactivated PrtR-PrtK within the oral cavity will not be toxic to the oral mucosal tissues. However, the inactivated PrtR-PrtK will stimulate the mucosal-associated lymphoid tissues (MALT) to produce specific antibody to neutralise the PrtR-PrtK of P. gingivalis.

Successful expression of a protein or peptide requires that either the insert comprising the gene or gene fragment, or the vector itself, contain the necessary elements for transcription and translation which is compatible with, and recognized by the particular host system used for expression. A variety of host systems may be utilized to express the PrtR-PrtK, which include, but are not limited to bacteria transformed with a bacteriophage vector, plasmid vector, or cosmid DNA; yeast containing yeast vectors; fungi containing fungal vectors; insect cell lines infected with virus (e.g. baculovirus); and mammalian cell lines transfected with plasmid or viral expression vectors, or infected with recombinant virus (e.g. vaccinia virus, adenovirus, adeno-associated virus, retrovirus, etc.).

Using methods known in the art of molecular biology various promoters and enhancers can be incorporated into the vector or the DNA sequence encoding PrtR-PrtK to increase the expression of the PrtR-PrtK amino acid sequences, provided that the increased expression of the amino acid sequences is compatible with (for example, non-toxic to) the particular host cell system used. Further, the DNA can be fused to DNA encoding other antigens, such as other bacterial outer membrane proteins, or other bacterial, fungal, parasitic, or viral antigens to create a genetically fused (sharing a common peptide backbone) multivalent antigen for use as an improved vaccine composition.

The selection of the promoter will depend on the expression system used. Promoters vary in strength, i.e. ability to facilitate transcription. Generally, for the purpose of expressing a cloned gene, it is desirable to use a strong promoter in order to obtain a high level of transcription of the gene and expression into gene product. For example, bacterial, phage, or plasmid promoters known in the art from which a high level of transcription have been observed in a host cell system comprising E. coli include the lac promoter, trp promoter, recA promoter, ribosomal RNA promoter, the P_{R} and P_{L} promoters, lacUV5, ompF, bla, lpp, and the like, may be used to provide transcription of the inserted DNA sequence encoding PrtR-PrtK.

Additional, if PrtR-PrtK protein may be lethal or detrimental to the host cells, the host cell strain/line and expression vectors may be chosen such that the action of the promoter is inhibited until specifically induced. For example, in certain operons the addition of specific inducers is necessary for efficient transcription of the inserted DNA (e.g., the lac operon is induced by the addition of lactose or isopropylthio-beta-D-galactoside). A variety of operons such as the trp operon, are under different control mechanisms. The trp operon is induced when tryptophan is absent in the growth media. The p_{L} promoter can be induced by an increase in temperature of host cells containing a temperature sensitive lambda repressor. In this way, greater than 95% of the promoter-directed transcription may be inhibited in uninduced cells. Thus, expression of recombinant PrtR-PrtK protein may be controlled by culturing transformed or transfected cells under conditions such that the promoter controlling the expression from the inserted DNA encoding PrtR-PrtK amino acid sequences is not induced, and when the cells reach a suitable density in the growth medium, the promoter can be induced for expression from the inserted DNA.

Other control elements for efficient gene transcription or message translation include enchancers, and regulatory signals. Enhancer sequences are DNA elements that appear to increase transcriptional efficiency in a manner relatively independent of their position and orientation with respect to a nearby gene. Thus, depending on the host cell expression vector system used, an enhancer may be placed either upstream or downstream from the inserted DNA sequences encoding PrtR-PrtK amino acid sequences to increase transcriptional efficiency. These or other regulatory sites, such as transcription or translation initiation signals, can be used to regulate the expression of the gene encoding PrtR-PrtK. Such regulatory elements may be inserted into DNA sequences encoding PrtR-PrtK amino acid sequences or nearby vector DNA sequences using recombinant DNA methods described herein for insertion of DNA sequences

Accordingly, P. gingivalis nucleotide sequences containing regions encoding for PrtR-PrtK, can be ligated into an expression vector at a specific site in relation to the vector's promoter, control, and regulatory elements so that when the recombinant vector is introduced into the host cell the P. gingivalis-specific DNA sequences can be expressed in the host cell. For example, the PrtR-PrtK specific DNA sequences containing their own regulatory elements can be ligated into an expression vector in a relation or orientation to the vector promoter and control elements which will allow for co-expression of the PrtR and PrtK. The recombinant vector is then introduced into the appropriate host cells, and the host cells are selected, and screened for those cells containing the recombinant vector Selection and screening may be accomplished by methods known in the art including detecting the expression of a marker gene (e.g., drug resistance marker) present in the plasmid, immunoscreening for production of PrtR-PrtK specific epitopes using antisera generated to PrtR-PrtK specific epitopes, and probing the DNA of the host's cells for PrtR-PrtK specific nucleotide sequence using one or more oligonucleotides and methods described herein.

Genetic engineering techniques may also be used to characterize, modify and/or adapt the encoded PrtR-PrtK protein. For example, site-directed mutagenesis to inactivate the protease domains of the PrtR-PrtK and to modify the protein in regions outside the protective domains, may be desirable to increase the safety and solubility.

In particular the host organism for the vector containing the PrtR-PrtK genes and constructs can be a commensal inhabitant of the oral cavity; for example an inhabitant of subgingival plaque, supragingival plaque or a bacterium associated with the oral mucosa. Examples of commensal intra-oral bacteria would be Streptococcus species and Actinomyces species, eg. Streptococcus salivarius, Streptococcus sanguis, Actinomyces naeslundii. These organisms can be isolated from the periodontitis patient and then genetically engineered to express the inactivated PrtR-PrtK. The DNA encoding the PrtR-PrtK could be linked with DNA encoding leader sequences of extracellular proteins of these commensal intra-oral bacteria. The DNA encoding the PrtR-PrtK could also be linked with, or inserted into, the DNA encoding extracellular proteins to produce secreted fusion proteins. Examples of extracellular proteins that could be used to produce fusion proteins with the inactivated PrtR-PrtK could be the glucosyltranferases (GTF) or fructosyltransferases (FTF). The recombinant organism would be then re-introduced into the patients oral cavity and once colonised the oral mucosa or teeth would express the inactivated PrtR-PrtK to stimulate the mucosal associated lymphoid tissue to produce neutralising antibodies.

Due to the conservation of the genes encoding PrtR-PrtK, the nucleic acid sequences of the present invention can be used in molecular diagnostic assays for detecting P. gingivalis genetic material. In particular, PrtR-PrtK sequence-specific oligonucleotides can be synthesized for use as primers and/or probes in amplifying, and detecting amplified, nucleic acids from P. gingivalis. Recent advances in molecular biology have provided several means for enzymatically amplifying nucleic acid sequences. Currently the most commonly used method, PCR™ (polymerase chain reaction Cetus Corporation) involved the use of Taq Polymerase, known sequences as primers, and heating cycles which separate the replicating deoxyribonucleic acid (DNA) strands and exponentially amplify a gene of interest Other amplification methods currently under development include LCR™ (ligase chain reaction, BioTechnica International) which utilizes DNA ligase, and a probe consisting of two halves of a DNA segment that is complementary to the sequence of the DNA to be amplified; enzyme QB replicase (Gene-Trak Systems) and a ribonucleic acid (RNA) sequence template attached to a probe complementary to the DNA to be copied which is used to make a DNA template for exponential production of complementary RNA; and NASBA™ (nucleic acid sequence-based amplification, Cangene Corporation) which can be performed on RNA or DNA as the nucleic acid sequence to be amplified.

Nucleic acid probes that are capable of hybridization with specific gene sequences have been used successfully to detect specific pathogens in biological specimens at levels of sensitivity approaching 10³ - 10⁴ organisms per specimen (1990, Gene Probes for Bacteria, eds. Macario and deMacario, Academic Press). Coupled with a method that allows for amplification of specific target DNA sequences, species-specific nucleic acid probes can greatly increase the level of sensitivity in detecting organisms in a clinical specimen. Use of these probes may allow direct detection without relying on prior culture and/or conventional biochemical identification techniques. This embodiment of the present invention is directed to primers which amplify species-specific sequences of the genes encoding PrtR-PrtK of P. gingivalis, and to probes which specifically hybridize with these amplified DNA fragments. By using the nucleic acid sequences of the present invention and according to the methods of the present invention, as few as one P. gingivalis organism may be detected in the presence of 10 ug/ml extraneous DNA.

DNA may be extracted from clinical specimens which may contain P. gingivalis using methods known in the art. For example, cells contained in the specimen may be washed in TE buffer and pelleted by centrifugation. The cells then may be resuspended in 100 ul of amplification reaction buffer containing detergents and proteinase K. Using the polymerase chain reaction, the resultant sample may be composed of the cells in 10 mM Tris pH 8.3, 50 mM KCl, 1.5 mM MgCl₂, 0.01% gelatin, 0.45% NP40™, 0.045% Tween 20™, and 60 ug/ml proteinase K. The sample is incubated in a 55° C. water bath for 1 hour. Following the incubation, the sample is incubated at 95° C. for 10 minutes to heat-inactivate the proteinase K. The sample may then be amplified in accordance with standard PCR protocols.

The following examples are further illustrative of the nature of the present invention, but it is understood that the invention is not limited thereto. All amounts and proportions referred to herein are by weight unless otherwise indicated

### EXAMPLE 1

### (1) Preparation of Antigen

### A. Anion exchange and affinity chromatography

P. gingivalis W50 was grown anaerobically at 37° C. on lysed horse blood agar and in modified BM media containing 1 μg/ml hemin. Bacteria were maintained on lysed horse blood plates by routine passage (<10 passages) and used to inoculate batch cultures. Batch culture growth in Brain Heart Infusion medium was monitored at 650 nm using a spectrophotometer (295E, Perkin-Elmer). Culture purity was checked routinely by Gram stain, microscopic examination and by using a variety of biochemical tests. Stocks were maintained as lyophilised cultures. A culture of P. gingivalis was grown to late logarithmic phase and the cells harvested by centrifugation (5,000×g, 20 min, 4° C.) and then resuspended in 160 ml TC buffer (20 mM Tris-HCl pH 7.4 and 5 mM CaCl₂) containing 50 mM NaCl and subjected to mild sonication using a Branson Sonifier 250 with an output control of 3 and a 50% duty cycle for 15 min at 4° C. The sonicate was centrifuged (100,000×g, 30 min, 4° C.) and the supernatant filtered (0.22 μm) prior to anion-exchange FPLC. The sonicate was applied to an anion-exchange column (Hiload XK 16/10 Q Sepharose, Pharmacia-LKB) cooled to 4° C., in multiple injections using a 50 ml superloop (Pharmacia-LKB). The sample was eluted using a linear gradient from 0 - 100% buffer B over 90 min at a flow rate of 2.0 ml min⁻¹. The eluant was monitored at 280 nm and collected in 6 ml fractions using a Frac 100 fraction collector (Pharmacia-LKB). Buffer A was TC buffer containing 50 mM NaCl and buffer B was TC buffer containing 500 mM NaCl Fractions were analysed for proteolytic and amidolytic activity using azocasein (A-2765, Sigma Chemical Co St Louis, Mo.), benzoyl-L-Arg-p-nitroanilide (BZ-L-Arg-pNa, Sigma) and benzyloxycarbonyl-L-Lys-p-nitroanilide (Z-L-Lys-pNa, Calbiochem, Melbourne, Australia) vide infra. Anion-exchange fractions containing the majority of proteolytic/amidolytic activity were pooled, washed and then concentrated in TC buffer containing 150 mM NaCl using a centricon 10 micro-concentrator (Amicon). The sample was then divided into four aliquots and each was independently applied to a gel filtration column (Superose 12, HR 10/30, Pharmacia-LKB) using TC buffer containing 150 mM NaCl at a flow rate of 0.3 ml min⁻¹. The eluant was monitored at 280 nm and peaks collected using a Frac 100 fraction collector The Mᵣ values of eluant peaks were determined using molecular mass gel filtration standards (Pharmacia-LKB). The peak containing the majority of the proteolytic/amidolytic activity was concentrated using a centricon 10 micro-concentrator and then applied at a flow rate of 0.1 ml min⁻¹ to an Arg-sepharose column (5 ml arginine-Sepharose 4B beads, HR 5/5 column, Pharmacia-LKB) and the unbound material collected. The column was washed with 500 mM NaCl and re-equilibrated with TC buffer containing 50 mM NaCl. The column was first eluted with 200 mM lysine-HCl pH 7.4 in TC buffer containing 50 mM NaCl at a flow rate of 0.1 ml min⁻¹. This was followed by 750 mM lysine-HCl pH 7.4 in the same buffer. The column was then re-equilibrated with TC buffer containing 50 mM NaCl and then eluted with 200 mM arginine-HCl pH 7.4 in TC buffer containing 50 mM NaCl at a flow rate of 0.1 ml min⁻¹. The unbound material collected was then re-applied to the Arg-sepharose column and the elution steps repeated. This sequence was repeated until all proteolytic activity had bound to the column. The eluant was monitored at 280 nm and peaks collected using a Frac 100 fraction collector. The peaks eluted from the Arg-sepharose by 200 mM lysine and 200 mM arginine were equilibrated with TC buffer containing 50 mM NaCl and 1.0% octyl-β-D-glucopyranoside and then applied to a Mono Q (HR 5/5) anion-exchange column and eluted using a linear gradient of 0 - 100% buffer B at a flow rate of 1.0 ml min⁻¹. Buffer A was TC buffer containing 50 mM NaCl and 0.1% octyl-β-D-glucopyranoside and buffer B was TC buffer containing 500 mM NaCl and 0.1% octyl-β-D-glucopyranoside. The eluant was monitored at 280 nm and eluant peaks collected using a Frac 100 fraction collector.

Azocasein, BZ-L-Arg-pNa and z-L-lys-pNa were used to routinely assay FPLC fractions for proteolytic and amidolytic activity. A sample of each fraction (20 - 200:1) was incubated at 37° C. with azocasein (5 mg/ml final concentration) in TC buffer pH 8.0 containing 150 mM NaCl and 10 mM cysteine. For azocasein the reaction was stopped by the addition of 30% trichloroacetic acid at 4° C. Samples were centrifuged and the A₄₄₀ of the supernatant measured using a spectrophotometer (Perkin Elmer, model 552).

For the synthetic chromogenic substrates samples of each chromatographic fraction (5 -50:1) were incubated at 37° C. with BZ-L-Arg-pNa or z-L-Lys-pNa (1.0 mM final concentration) in a total volume of 350:1 100 mM Tris-HCl pH 8.0 buffer containing 150 mM NaCl, 10 mM cysteine and 5 mM CaCl₂. Inhibitors and activators were added to the purified enzymes in 100 mM Tris-HCl pH 8.0 buffer containing 150 mM NaCl. Absorbance was measured at 410 nm in a Hewlett Packard 8452A Diode Array spectrophometer and the amidolytic activity expressed in U, where U=μmol substrate converted min⁻¹ at 37° C. Trypsin (E.C.3.4.21 4, T 8253 Sigma) was used as a standard. The protein concentration of FPLC fractions and purified samples was determined using the Bradford protein assay (Biorad) with BSA as a standard.

A sample of the gel filtration chromatographic fraction (20 μl) exhibiting the major proteolytic and amidolytic activity was incubated for 4 h at 37° C. with 10 mg/ml of pure αₛ₁-casein dissolved in TC buffer pH 8.0 containing 150 mM NaCl and 50 mM 2-mercaptoethanol. Following incubation the sample was equilibrated in 0.1% TFA (v/v) dissolved in Milli Q water (Buffer A). The sample was then applied to an HPLC reversed phase analytical column (C8, 7 μm, 4.6 mm×220 mm, Applied Biosystems Inc. Brownlee Aquapore RP 300) and peptides eluted using a linear gradient from 0 -100% buffer B over 40 min at a flow rate of 1 ml min¹ (140A solvent delivery system). Buffer B was 80% acetonitrile (v/v) in 0.1% (v/v) TFA in Milli Q water. The eluant was monitored at 214 nm using a 1000S diode array detector (Applied Biosystems). Peaks were collected manually and peptides identified using a combination of amino acid composition and sequence analyses as described previously.

SDS-PAGE was performed using a Mini protean II electrophoresis system (Biorad) with 12% (w/v), 1 mm separating gels, overlaid with 5% stacking gels (Laemmli, 1970) [Nature 277:680-685]. Two volumes of each sample were mixed with one volume of buffer [0.5 M Tris-HCl, pH 6.8, 5% v/v 2-mercaptoethanol, 10.0% w/v SDS, 0.05% w/v bromophenol blue (75% v/v) and glycerol (25% v/v)] and heated to 100° C. for 4 min unless otherwise stated. SDS-PAGE was performed at room temperature using a current of 30 - 50 mA and a potential difference of ≦200 V. For silver staining, gels were fixed in methanol/water/acetic acid (45/45/10, v/v/v), washed in Milli Q water, reduced with 5 μg/ml dithiothreitol and then washed in Milli Q water, all for 30 min periods. Gels were then stained for 20 min with 0.1% w/v AgNO₃ and developed with 3% w/v sodium carbonate containing 0.1% v/v formaldehyde and development stopped with glacial acetic acid. For Coomassie blue staining, gels were fixed in 12% TCA and stained overnight using 0.1% (w/v) purified Coomassie brilliant blue G 250 in 2% (w/v) phosphoric acid, 6% (w/v) ammonium sulphate. Gels were destained with methanol/water/acetic acid (50/40/10, v/v/v). Proteins were transferred onto a PVDF membrane (Problott, Applied Biosystems Inc. (ABI)) for sequence analysis using a transblot cell (Biorad). PVDF membrane was wetted in 100% methanol and soaked in transfer buffer (10 mM CAPS/10% methanol, pH 11.5). Transfer was performed using a potential difference of 60 V (300 mA) for 90 min. Membranes were briefly stained using 0 1% (w/v) Coomassie brilliant blue R 250 in methanol/water/acetic acid (5/5/1, v/v/v). Protein bands were excised, destained for 10 - 30 sec in 50% methanol and then the N-terminal sequence determined using a Hewlett Packard 10005A protein sequencer or a modified ABI 471-02A protein sequencer fitted with a blott cartridge.

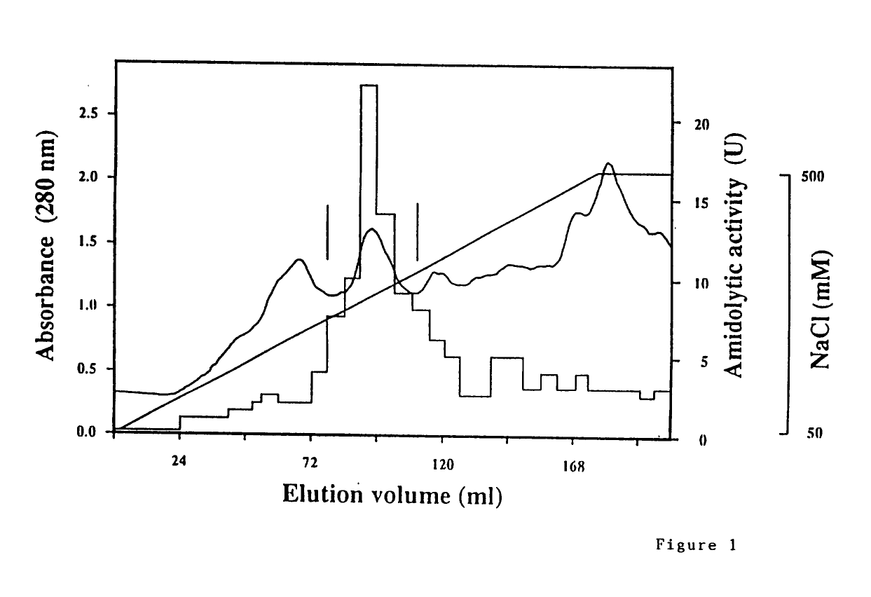

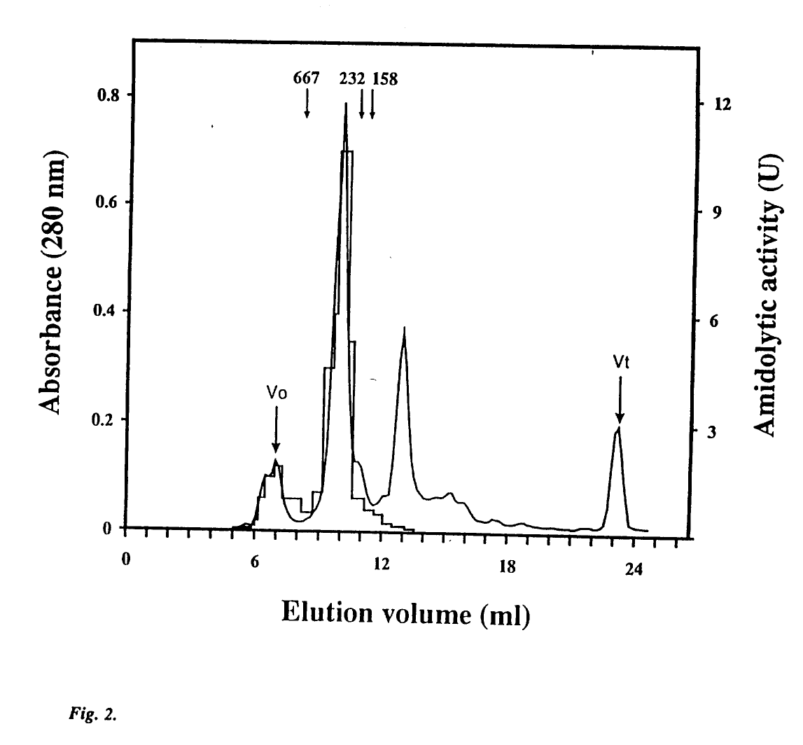

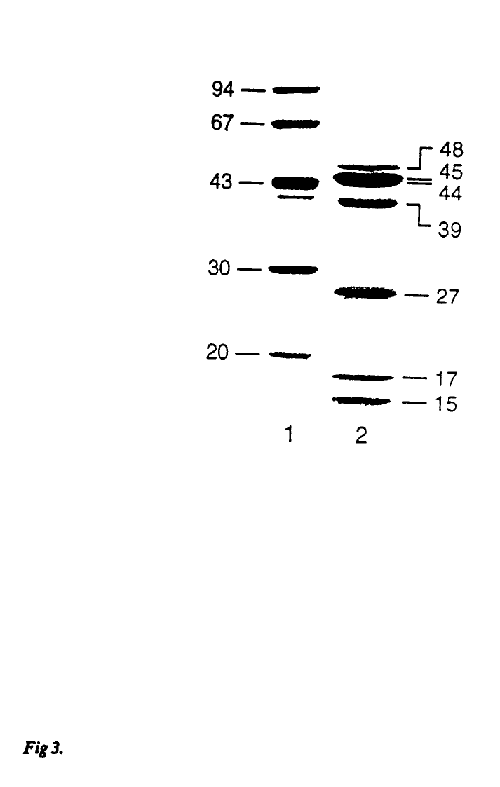

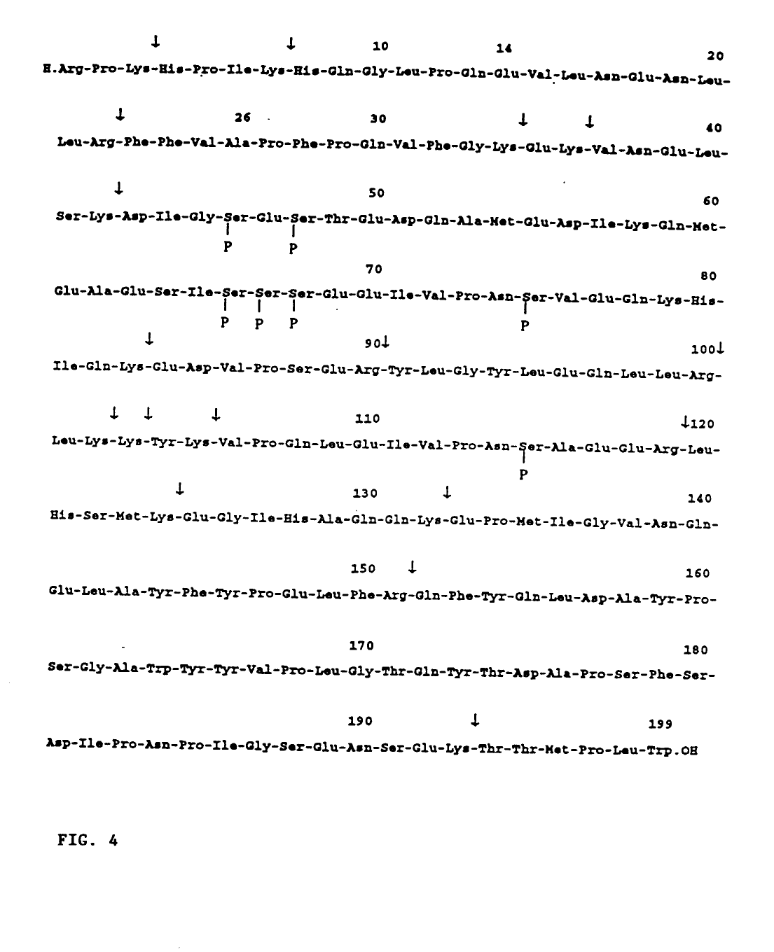

The ultrasonication procedure was effective at releasing the cell-associated Arg- and Lys-specific proteolytic activity of P. gingivalis W50 and 15 min was required for maximal release of activity. The sonicate of P. gingivalis W50 cells contained 0.30 mg ml⁻¹ protein and 2.6 and 2.3 μmol min⁻¹ mg protein⁻¹ activity with 1.0 mM Bz-L-Arg-pNA and z-L-Lys-pNA as substrate respectively at 37° C. The crude sonicate was subjected to Q-sepharose anion exchange FPLC and a representative chromatogram is presented in FIG. 1. Proteolytic/amidolytic activity eluted as one major peak between 246 - 320 mM NaCl (FIG. 1) which was collected, concentrated using a centricon-10 (Amicon) and then applied to the Superose 12 gel filtration column (FIG. 2). Molecular mass gel filtration standards were used to determine the Mᵣ of the peaks obtained and the major peak, which also exhibited the major proteolytic/amidolytic activity, corresponded to 300 kDa (FIG. 2). Proteolytic/amidolytic activity was also associated with the high molecular mass material (0.6 ->2.0×10⁶ Da) eluted from the gel filtration column. The 300 kDa gel filtration peak contained seven bands at 48, 45, 44, 39, 27, 17 and 15 kDa on SDS-PAGE analysis (FIG. 3). The seven bands were transblotted and subjected to N-terminal sequence analysis (Table 1). This analysis revealed that the 44 kDa band contained two proteins and the N-terminal sequences of these two 44 kDa proteins were assigned after further purification. The N-terminal sequence of one of the 44 kDa proteins was identical to that of the 17 kDa protein and the 39 kDa and 27 kDa proteins also had identical N-termini (Table 1).

TABLE 1

N-terminal sequences of proteins in the 300 kDa
complex separated by SDS-PAGE

| Band | N-terminal sequence (kDa) |
|---|---|
| 48* | DVYTDHGDLYNTPVRML |
| 45† | YTPVEEKQNGRMIVIVAKKYEGD |
| 44† | SGQAEIVLEAHDVWNDGSGYQILLDADHDQYGQVIPSDTHFL |
| 44* | PQSVWLERTVDLPAGTKYVAFR |
| 39* | ANEAKVVLAADNVWGDNTGYQFLLDA |
| 27† | ANEAKVVLAADNVWGDNTGYQFLLDA |
| 17† | PQSVWIERTVDLPAGTKYVAFR |
| 15*† | ADFTETFESSTHGEAPAEWTTIDA |

* Proteins eluted from Arg-sepharose by 200 mM lysine
†Proteins eluted from Arg-sepharose by 200 mM arginine.

Repeated gel filtration analyses of the Q-sepharose purified material or crude sonicates indicated that the major proteolytic/amidolytic activity was associated with a peak corresponding to 300 kDa and higher molecular mass (0.6 ->2×10⁶ Da) material that when boiled in SDS and subjected to SDS-PAGE analysis contained the same seven bands at 48, 45, 44, 39, 27, 17 and 15 kDa (FIG. 3).

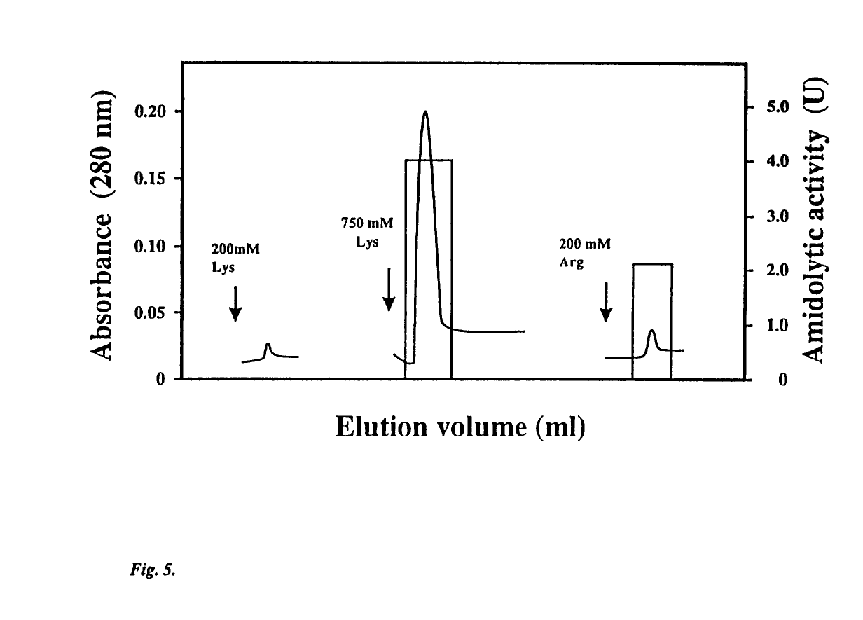

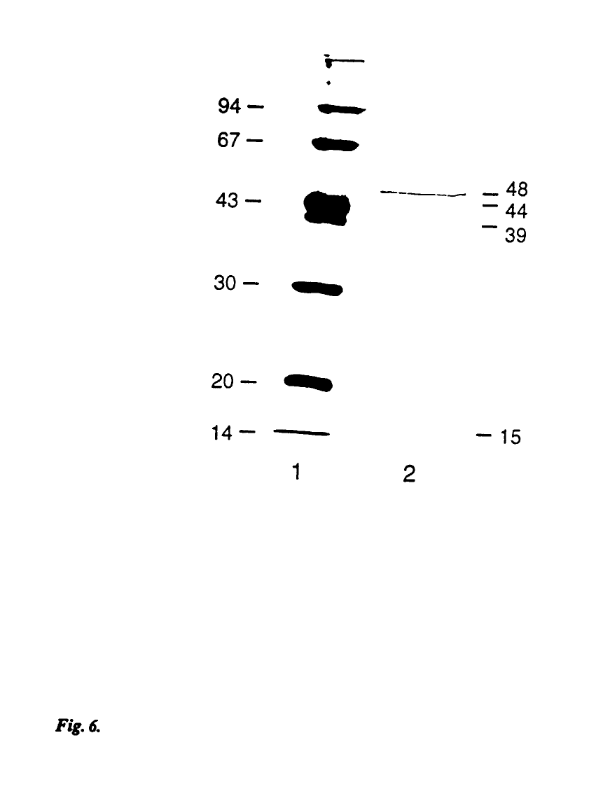

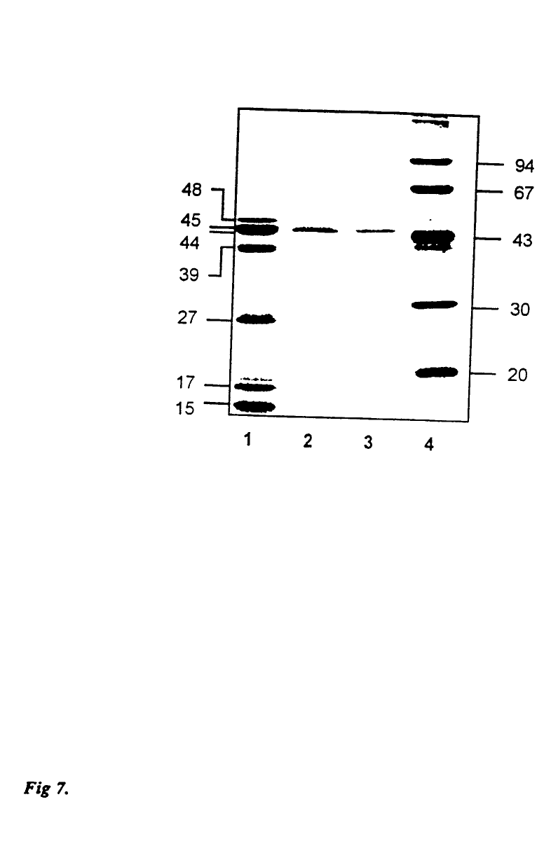

The 300 kDa gel filtration protein complex was incubated with αₛ₁-casein. The αₛ₁-casein peptides released by the action of the proteolytic activity of the 300 kDa complex were purified by RP-HPLC and identified by amino acid composition and sequence analyses. The sites of αₛ₁-casein cleavage by the material of the 300 kDa complex were the carboxyl side of arginyl and lysyl residues only (FIG. 4). All arginyl and lysyl residues of αₛ₁-casein were cleaved except the N-terminal Arg and the Lys residues flanking the Ser(P) cluster sequence, presumably due to the high negative charge density (FIG. 4). The 300 kDa complex was then applied to an Arg-sepharose column and washed with TC buffer containing 500 mM NaCl (FIG. 5). The Arg-sepharose was eluted first with 200 mM lysine in TC buffer (FIG. 5) which eluted a small amount of the 48 kDa, 44 kDa, 39 kDa and 15 kDa proteins of the 300 kDa complex as shown by SDS-PAGE (FIG. 6 and Table 1). N-terminal sequence analysis of these transblotted proteins revealed that only one of the 44 kDa proteins of the 300 kDa complex was eluted with 200 mM lysine (Table 1). This fraction eluted from Arg-sepharose with 200 mM lysine contained only Lys-specific proteolytic/amidolytic activity. Next the Arg-sepharose column was eluted with 750 mM lysine (FIG. 5) which removed the majority of the protein bound as the undissociated 300 kDa complex containing all seven bands (eight proteins) as shown by SDS-PAGE analysis (FIG. 7). The 750 mM lysine eluant exhibited both Arg- and Lys-specific proteolytic/amidolytic activity characteristic of the 300 kDa complex. The Arg-sepharose column was then eluted with 200 mM arginine in TC buffer (FIG. 5). The 200 mM arginine eluant contained small amounts of the 45, 44, 27, 17 and 15 kDa proteins as shown by SDS-PAGE (FIG. 7). This fraction exhibited only Arg-specific proteolytic/amidolytic activity. N-terminal sequence analysis of these transblotted proteins eluted with 200 mM arginine revealed that only one of the 44 kDa proteins of the 300 kDa complex was eluted with 200 mM arginine and this 44 kDa protein was different to the 44 kDa protein eluted with 200 mM lysine (Table 1).

The proteins eluted from the Arg-sepharose column with 200 mM lysine and 200 mM arginine were washed, concentrated and equilibrated with TC buffer containing 50 mM NaCl and 1.0% octyl-β-D-glucopyranoside and applied independently to a Mono Q anion exchange column. Elution from the Mono Q column with a NaCl gradient associated the Arg-specific proteolytic activity with the 45 kDa protein with a 25 fold purification over the original crude sonicate (Table 2, FIG. 7). The specificity of the 45 kDa proteinase for arginyl residues was confirmed by the enzyme cleaving Bz-L-Arg-pNA but not z-L-Lys-pNA. The Arg-specific 45 kDa enzyme was activated by thiols (particularly cysteine), not inhibited by PMSF or AEBSF but inhibited by sulphydryl-directed reagents, leupeptin and EDTA (Table 3). The inhibition by EDTA could be reversed by the addition of Ca²⁻ (Table 3) The pH optimum of the enzyme was 7.5-8.0 and activity dropped off dramatically as the pH was lowered below 7.0. These results indicate that the 45 kDa enzyme is a calcium-stabilized, Arg-specific cysteine endopeptidase. The Lys-specific activity was characterized using the substrate Z-L-Lys-pNA and was associated with the 48 kDa protein purified from the 200 mM lysine eluant by Mono Q FPLC The Lys-specific enzyme was also activated by thiols and inhibited by sulphydryl-directed reagents but was not inhibited by leupeptin or EDTA. Non-reducing SDS-PAGE without boiling of the 300 kDa complex produced bands corresponding to the relative molecular masses of approximately 300, 150, 104, 88, 76 and 66 kDa.

TABLE 2
Purification of the 45 kDa Arg-specific proteinase PrtR45

| | | Proteolytic | Specific | | |
|---|---|---|---|---|---|
| | Protein | activity | activity | Purification | Yield |
| Step | (mg) | (U*) | U mg⁻¹ | fold | % |
| Sonicate | 48.0 | 124 | 2.6 | 1 | 100 |
| Anion Exchange | 8.2 | 64 | 7.8 | 3 | 52 |
| FPLC (Q-sepharose) | | | | | |
| Gel filtration FPLC | 3.9 | 46 | 11.8 | 5 | 37 |
| (Superose 12) | | | | | |
| Affinity FPLC | 0.7 | 17 | 24.3 | 9 | 14 |
| (Arg-sepharose) | | | | | |
| Anion exchange | 0.2 | 13 | 65.0 | 25 | 11 |
| FPLC | | | | | |
| (mono Q) | | | | | |

*Amidolytic activity using 1.0 mM Bz-L-Arg-pNA; 1 unit = μmol min⁻¹ at 37° C.

TABLE 3
Effects of various activators/inhibitors on the activity of the 45 kDa
Arg-specific proteinase

| | Concentration | Activity |
|---|---|---|
| Compound | (mM) | (%) |
| 2-mercaptoethanol | 1.0 | 100 |
| | 10.0 | 158 |
| | 50.0 | 189 |
| Dithiothreitol | 1.0 | 109 |
| | 10.0 | 174 |
| L-cysteine | 0.1 | 183 |
| | 1.0 | 320 |
| | 10.0 | 487 |
| PMSF*^{,†} | 1.0 | 100 |
| | 10.0 | 90 |
| AEBSF*^{,†} | 1.0 | 93 |
| | 10.0 | 80 |
| Iodoacetic acid^{†} | 1.0 | 82 |
| | 10.0 | 19 |
| PCMB*^{,†} | 1.0 | 100 |
| | 10.0 | 14 |
| Leupeptin^{†} | 0.1 | 0 |
| EDTA^{†} | 1.0 | 100 |
| | 10.0 | 4 |
| | 50.0 | 0 |
| +Ca²⁺ | 50.0 | 97 |
| o-phenanthroline^{†} | 10.0 | 100 |

*PCMB, p-chloromercuribenzoic acid; PMSF, phenylmethyl sulfonyl fluoride; AEBSF, [4-(2-aminoethyl)-benzenesulfonylfluoride]
^{†}These incubations also contained 1.0 mM 2-mercaptoethanol

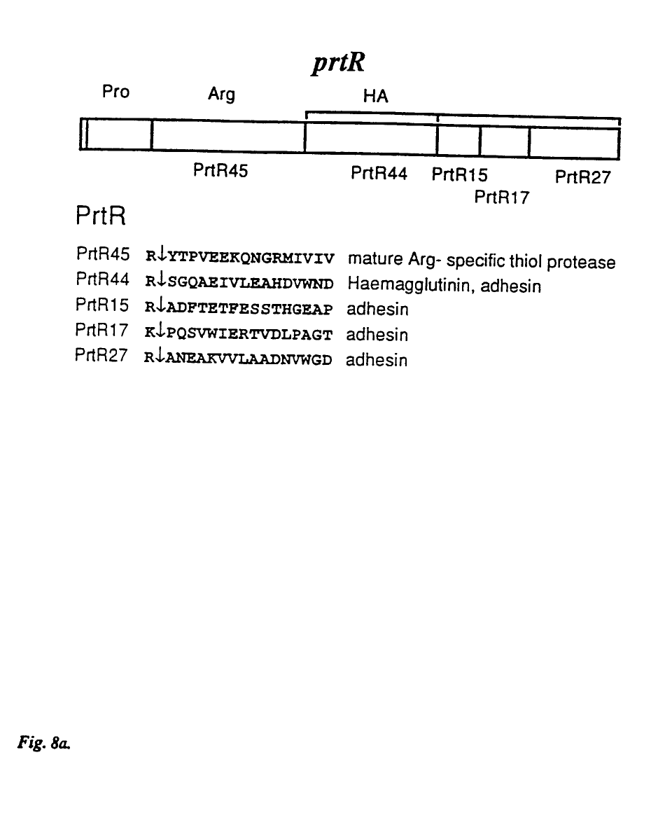

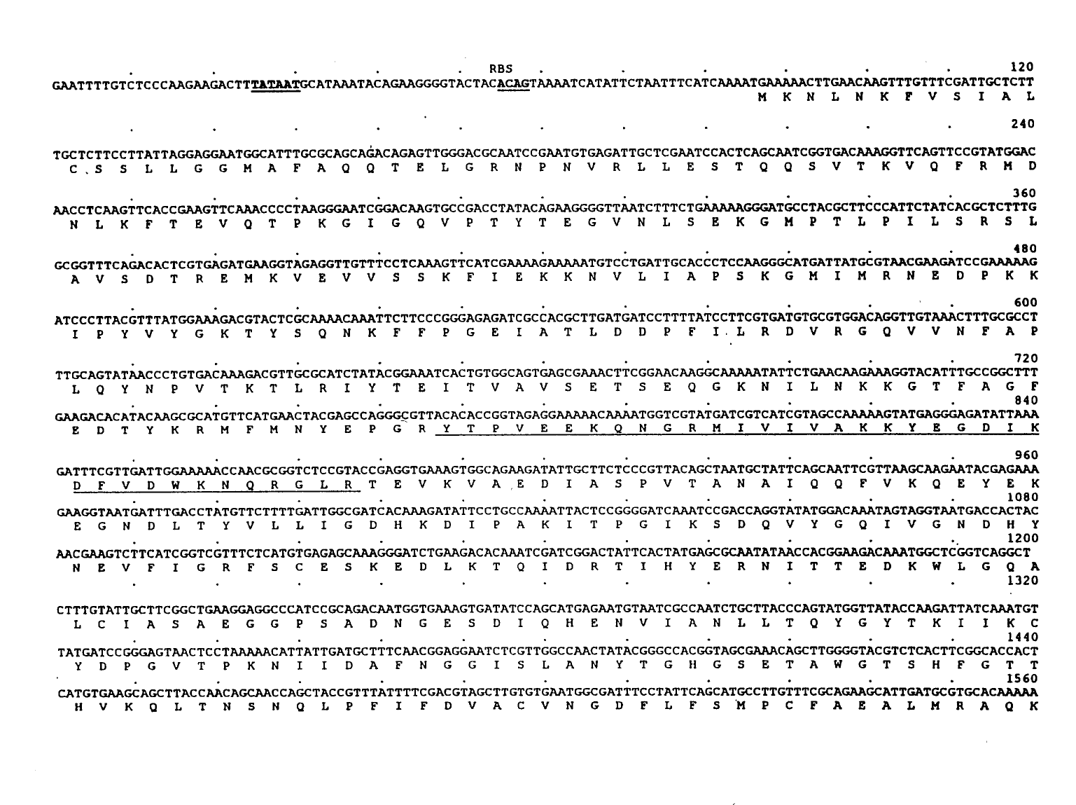

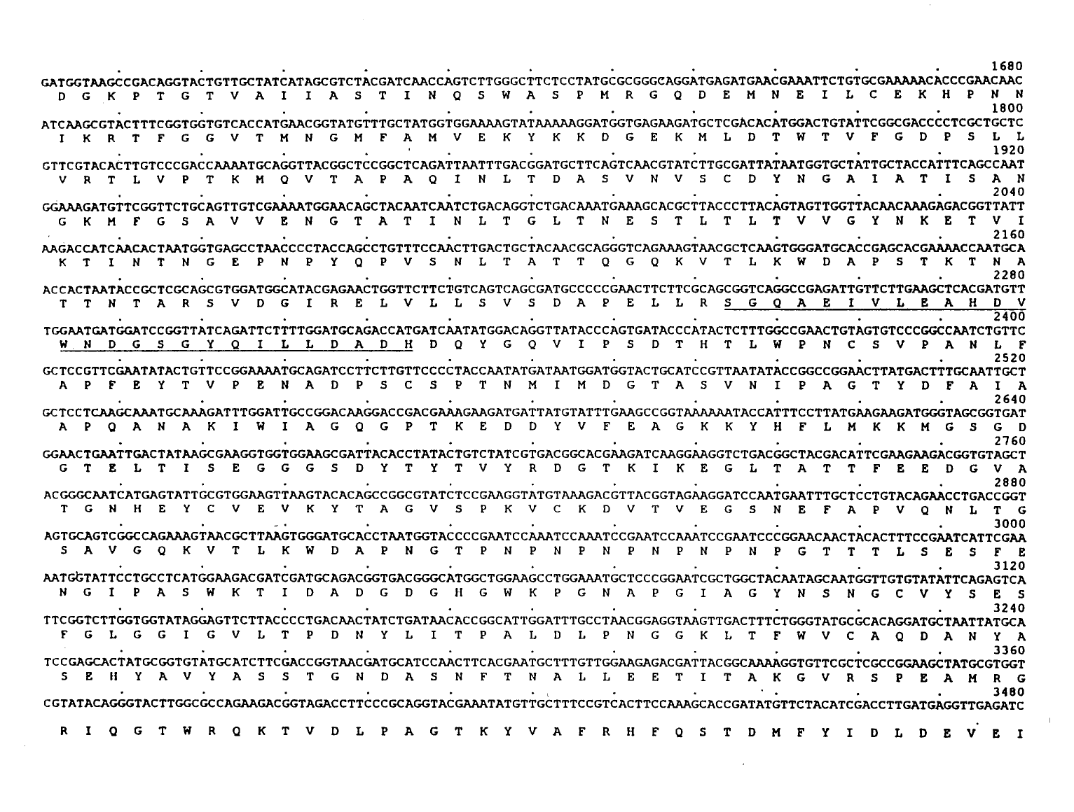

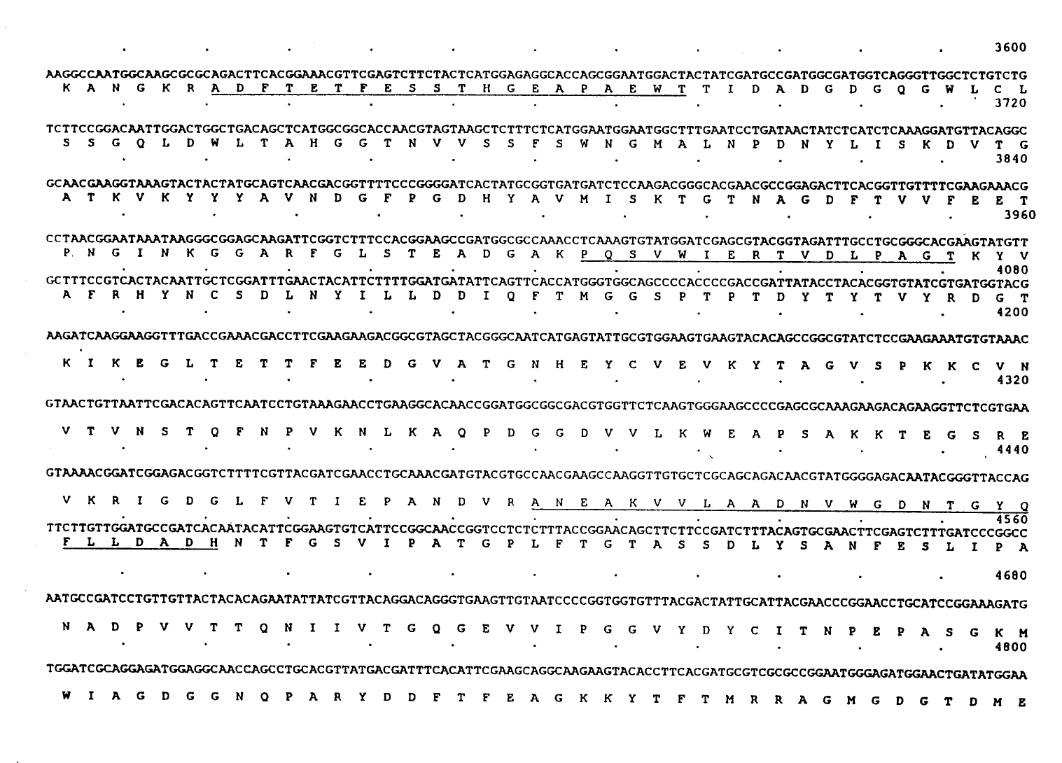

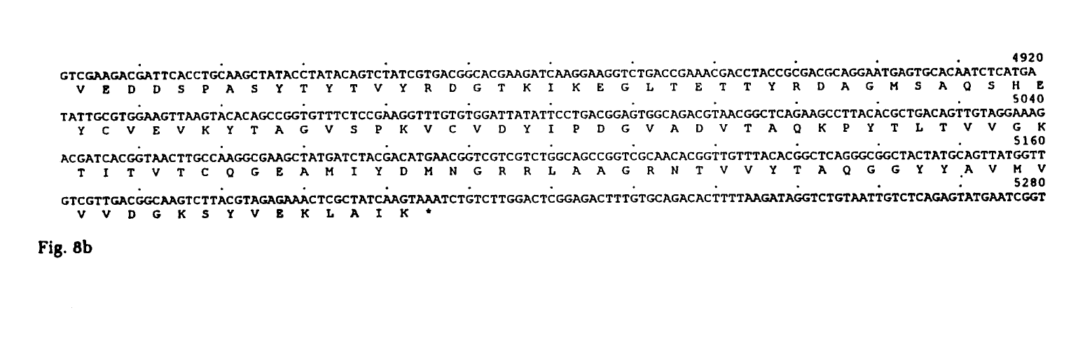

The 45, 27, 17, 15 kDa and one of the 44 kDa protein components of the 300 kDa complex are encoded by the gene the PrtR as presented schematically in FIG. 8a. The complete nucleotide sequence and deduced amino acid sequence of the PrtR is shown in FIG. 8b. Each PrtR component is preceded by an arginyl or lysyl residue (FIGS. 8a, b) indicating that the polyprotein is processed by trypsin-like proteolytic specificity. We have designated these component parts of the 300 kDa complex, by their relative molecular masses as determined by SDS-PAGE, as the PrtR45, PrtR44, PrtR27, PrtR17 and PrtR15 which fit well with the predicted sizes from the deduced PrtR amino acid sequence (53.9, 44.8, 29.5, 17.5 and 14.3 kDa respectively). The 44 kDa protein, the PrtR44, has been disclosed by previous workers as a culture fluid hemagglutinin/adhesin (Pike et al., 1994)[J Biol Chem 269:406-411]. The PrtR44 has homology with the other non-proteinase components of the multiprotein complex suggesting a similar role for the PrtR27, PrtR17 and PrtR15 in interacting with the protease and/or in hemagglutination or adhesion. The PrtR45 Arg-specific endopeptidase component of the PrtR complex has the same characteristics and N-terminal sequence as the 50 kDa Arg-specific proteinase identified in the culture supernatant of P. gingivalis H66 by Chen et al. (1992)[J Biol Chem 267:18896-18901] designated Arg-gingipain.

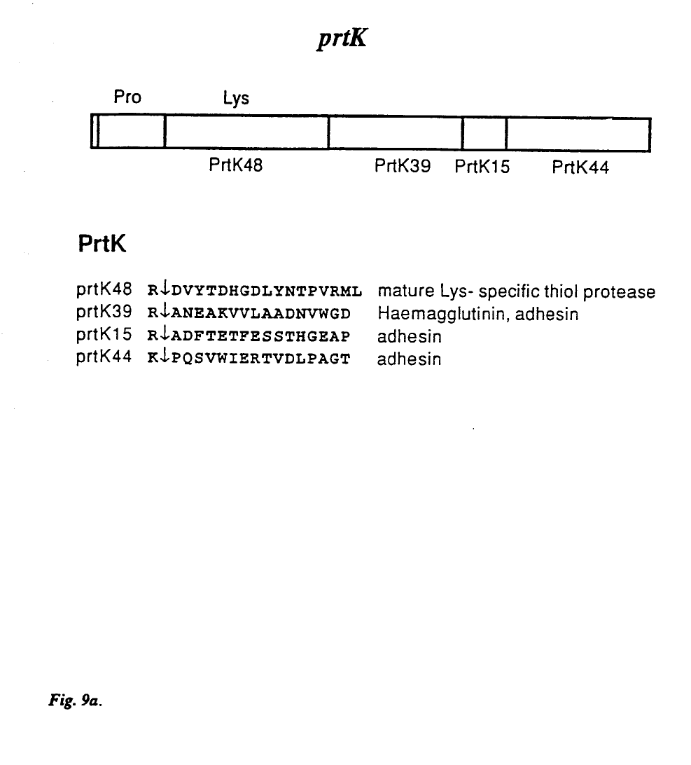

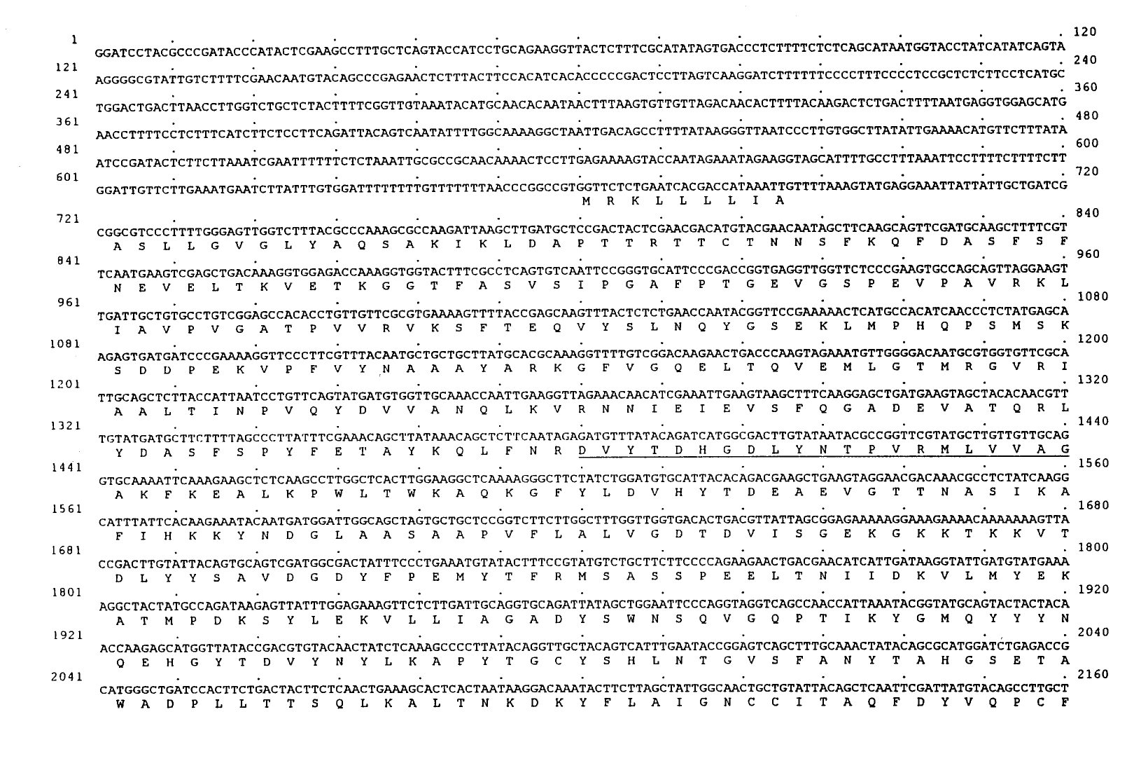

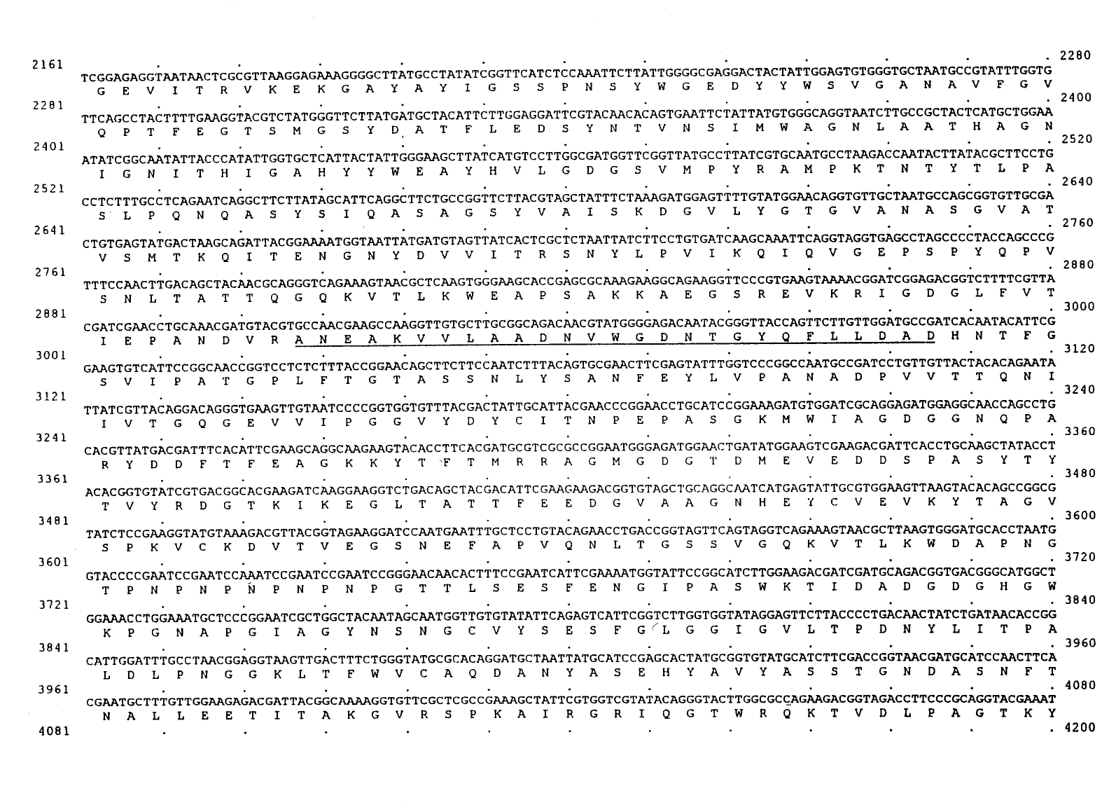

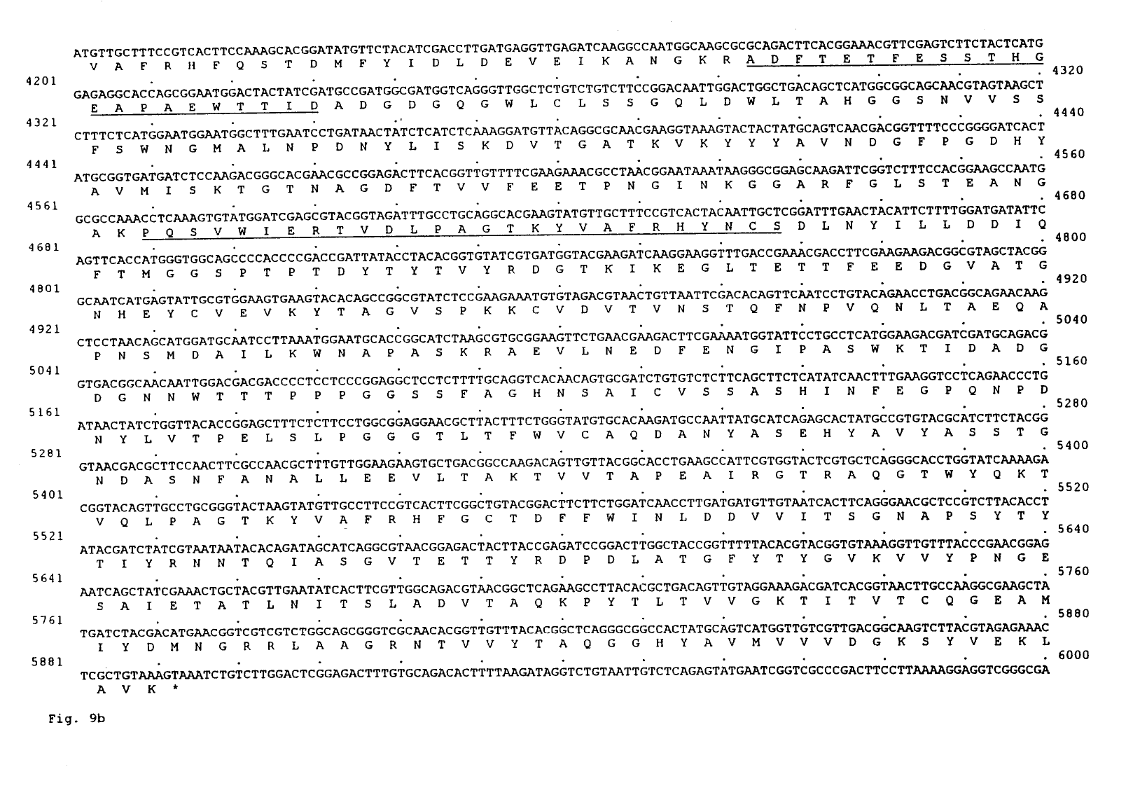

The other proteins of the 300 kDa complex, the 48 kDa Lys-specific proteinase, the other 44 kDa protein and the 39 kDa and 15 Da proteins are encoded by a single gene the prtK presented schematically in FIG. 9a. The complete nucleotide sequence and deduced amino acid sequence of the PrtK is shown in FIG. 9b. The prtK is similar to the prtR in that it encodes a putative leader sequence, a prosequence followed by the proteinase domain which is then followed by sequence-related adhesins that have high homology with the C-terminal adhesins of the prtR. We have designated the 48 kDa Lys-specific proteinase the PrtK48 and its associated adhesins the PrtK39, PrtK15 and PrtK44 (FIGS. 9a, b) based on the sizes measured by SDS-PAGE which fit reasonably well with the predicted sizes from the deduced PrtK amino acid sequence (55.9, 44.8, 14.3 and 47.9 kDa respectively). The PrtK48 has the same enzyme characteristics as the 48 kDa proteinase purified from the culture supernatant of P. gingivalis 33277 by Fujimura et al. (1993) [Infect Immun 55.716-720]. The PrtK48 also has the same N-terminal sequence and enzyme characteristics as the 60 kDa Lys-specific endopeptidase previously purified from the culture fluid of P. gingivalis H66 by Pike et al. (1994) [J Biol Chem 269:406-411] and designated Lys-gingipain. The PrtK39, PrtK15 and PrtK44 are all sequence-related and have high homology with the PrtR hemagglutinins/adhesins particularly the 15 kDa protein which is identical in both gene products suggesting that these proteins also are hemagglutinin/adhesins.

As the 300 kDa proteinase-adhesin complex and higher molecular mass forms are composed of proteins from the two genes, the prtR and prtK, we suggest that they be designated PrtR-PrtK complexes. The deduced molecular mass of the mature PrtR is 160 kDa (FIGS. 9a, b) and mature PrtK is 163 kDa (FIG. 9b) such that the mass of the PrtR-PrtK heterodimer would be 323 kDa which is in good agreement with the Mᵣ determined by gel filtration and non-boiling SDS-PAGE. SDS-PAGE of the sample after boiling produced the seven bands of 48, 45, 44, 39, 27, 17 and 15 kDa corresponding to the domains of the two gene products, the PrtR and PrtK. These domains were only seen when the sample was boiled, with or without reducing agent, suggesting that the domains remain tightly non-covalently associated after proteolytic processing. The cell sonicate and the chromatographic fractions had minimal or no proteolytic activity in the absence of reducing agents thus ensuring minimal enzymic activity during the chromatographic purifications. The characterization of the 300 kDa cell-associated complex as being composed of processed domains of the two genes the prtR and prtK suggests that the secreted, mature PrtR and PrtK proteins associate and then are processed, perhaps autolytically The identification of several of the domains of the PrtR and PrtK in the culture supernatant by independent groups is consistent with the proteolytic (autolytic) processing of these polyproteins.

The relative molecular mass of the processed PrtR-PrtK complex is likely to be attributable to the composition of 1 PrtK48+1 PrtR45+1 PrtR44+1 PrtK39+1 PrtK44+1 PrtR27+1 PrtR17+1 PrtK15+1 PrtR15=294-323 kDa depending on C-terminal truncation, that is the 300 kDa complex would contain the five domains of the prtR and the four domains of the prtK gene products (FIGS. 8 and 9). As high Mᵣ material (0.6 ->2×10⁶ Da) on gel filtration (FIG. 2) was also composed of the seven PrtR-PrtK bands then this suggests that the 300 kDa PrtR-PrtK complexes may further associate to form larger cell-associated aggregates. The high amino acid sequence homology between the PrtR44, PrtK39, PrtK44, PrtR27, PrtR17 and the 15 kDa protein of both the PrtR and PrtK suggests that these adhesins are responsible for the non-covalent cohesive interactions between the components of the PrtR-PrtK complexes and between the complexes themselves in the larger aggregates. It is interesting to note that some dissociation of the 300 kDa PrtR-PrtK complex occurred during the affinity chromatography on Arg-sepharose, although the majority of the protein eluted as the undissociated complex with 750 mM lysine. The partial dissociation of the complex on binding to substrate may be a mechanism by which the complex targets specific host macromolecules and cells releasing the proteinase/adhesin domains at the target site on binding.

This example describes the purification of a novel cell associated complex of Arg-specific and Lys-specific proteinases and sequence-related adhesins encoded by the two genes, the prtR and prtK.

### B. Ultrafiltration and Diafiltration

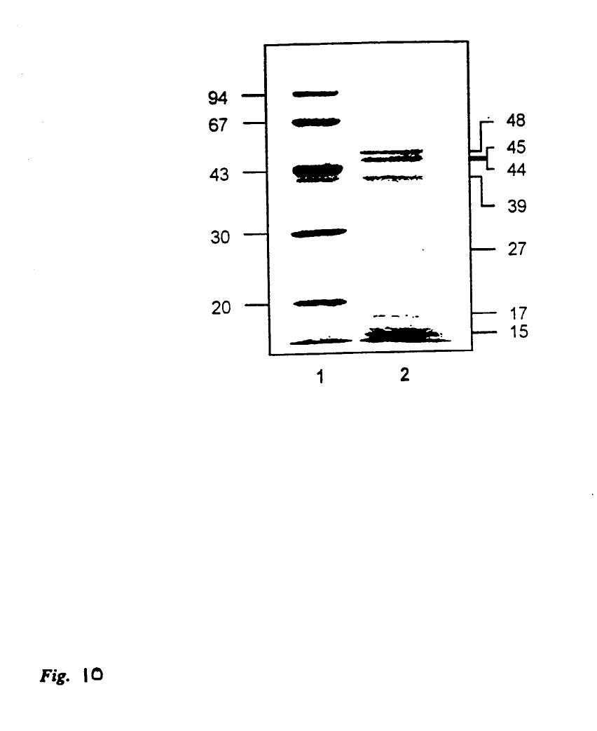

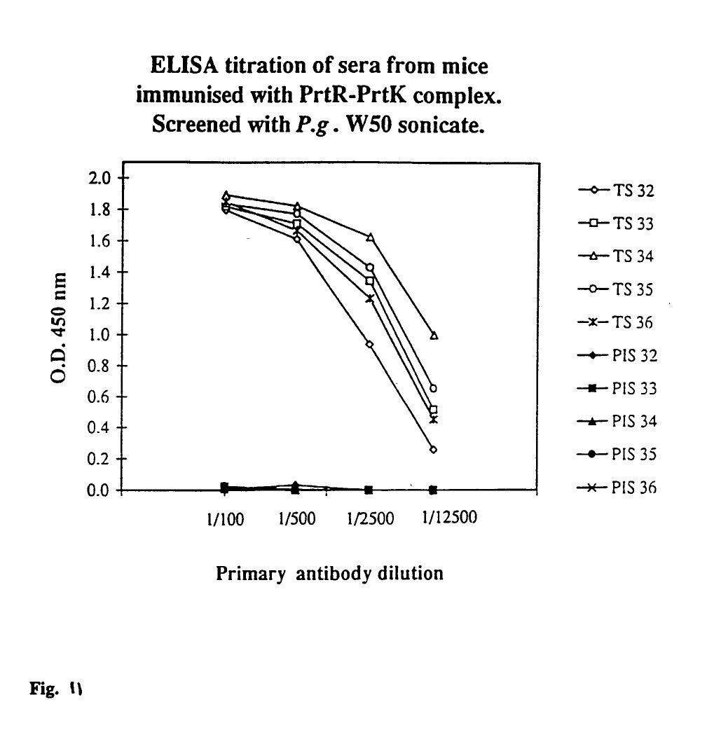

P. gingivalis W50 was grown anaerobically at 37° C. on lysed horse blood agar and in modified BM media containing 1 μg/ml hemin. Bacteria were maintained on lysed horse blood plates by routine passage (<10 passages) and used to inoculate batch cultures. Batch culture growth in Brain Heart Infusion medium was monitored at 650 nm using a spectrophotometer (295E, Perkin-Elmer). Culture purity was checked routinely by Gram stain, microscopic examination and by using a variety of biochemical tests. Stocks were maintained as lyophilised cultures. A culture of P. gingivalis was grown to late logarithmic phase and the cells harvested by centrifugation (5,000×g, 20 min. 4° C.). Chloroform was added to the cell pellet and after gentle mixing the suspension was left for 15 min at room temperature. Following chloroform treatment, 20 mM Tris-HCl pH 8.0 buffer containing 50 mM NaCl was added and gently mixed. This mixture was then centrifuged (100,000×g, 30 min, 4° C.) and the supernatant diafiltered through a 100,000 Mᵣ cut-off membrane (Amicon) with five volumes of distilled water. This purifies and inactivates by oxidation the 294-323 kDa PrtR-PrtK which is freeze dried and used as an immunogen. The PrtR-PrtK purified by diafiltration was composed of 48, 45, 44, 39, 27, 17 and 15 kDa components as shown by SDS-PAGE (FIG. 10).

### (2) Preparation of Antibodies

Polyclonal antiserum to PrtR-PrtK was raised in a rabbit by immunizing with the O₂-inactivated PrtR-PrtK subcutaneously The rabbit was immunized at day 0 with 40 μg of protein in incomplete Freund's adjuvant, day 14 with 90 μg of protein in incomplete Freund's adjuvant, and day 28 with 60 μg of protein in incomplete Freund's adjuvant. Immunizations were carried out using standard procedures. Polyclonal antisera having a high titre against P. gingivalis was obtained. If desired the antibodies directed specifically against P. gingivalis can be obtained using standard procedures.

### EXAMPLE 2

Methods and compounds for vaccine rormulations related to PrtR-PrtK.

This embodiment of the present invention is to provide PrtR-PrtK protein to be used in as an immunogen in a prophylactic and/or therapeutic vaccine for active immunization to protect against or treat infections caused by P. gingivalis. For vaccine purposes, an antigen of P. gingivalis comprising a bacterial protein should be immunogenic, and induce functional antibodies directed to one or more surface-exposed epitopes on intact bacteria, wherein the epitope(s) are conserved amongst strains of P. gingivalis.

In one illustration of the PrtR-PrtK protein having the properties desirable of a vaccine antigen, the protein was purified from P. gingivalis using the method described herein in Example 1. Mice were immunized with the purified inactivated PrtR-PrtK protein (25 μg) with adjuvant (20 μg of QS21) two times at four week intervals The purified PrtR-PrtK was inactivated by air oxidation. Blood from the immunized mice was drawn 32 days after the last immunization and the immune sera was pooled. The pooled immune sera was assayed against whole bacteria (P. gingivalis strain W50) by an enzyme linked immunosorbent assay (ELISA). For the whole cell ELISA, overnight cultures of bacteria were harvested by a swab and suspended in PBS to an absorbance of 0.1 at 600 nm. Aliquots (100 μl) of the bacterial suspension were added to the wells of a 96 well microtiter plate and dried overnight at room temperature. The plates were blocked with 100 μl of 0.1% (w/v) gelatin in PBS. This, and all remaining incubations, were for one hour at room temperature unless otherwise specified. The blocking solution was removed and 100 μl of the immune sera, diluted in PBS with 0.1% (w/v) gelatin, was added to the wells and incubated. After washing three times with PBS, the bound antibodies were detected by incubating with 100 μl of alkaline phosphatase conjugated recombinant protein G (1:1500 in PBS with 0.1% (w/v) gelatin). The plates were washed and colour development was facilitated by the addition of 100 μl/well of p-nitrophenyl phosphate (2 mg/ml in diethanolamine). After 30 minutes, the reaction was stopped by adding 50 μl of 3M NaOH. The absorbance was read at 492 nm using an ELISA reader. Endpoint titers were determined as the reciprocal of the dilution at which the absorbance was greater than that of the blank wells. The results demonstrated that immunization with inactivated PrtR-PrtK elicit antibodies which can bind to one or more surface-exposed epitopes on intact P. gingivalis.

Additional evidence supporting the immunogenicity of the PrtR-PrtK protein comes from a study of the human immune response to the PrtR-PrtK of P. gingivalis in which 86% of 43 patients with adult periodontitis had specific IgG in their sera to the PrtR-PrtK.

Another illustration of a desirable vaccine antigen is the O₂-inactivated PrtR-PrtK. It has been demonstrated that the cell surface PrtR-PrtK is the target of bactericidal antibody generated from immunization with the inactivated protein. Polyclonal antiserum to PrtR-PrtK was raised in a rabbit by immunizing with the inactivated PrtR-PrtK subcutaneously. A rabbit was immunized at day 0 with 40 μg of protein in incomplete Freund's adjuvant, day 14 with 90 μg of protein in incomplete Freund's adjuvant, and day 28 with 60 μg of protein in incomplete Freund's adjuvant. The resultant antiserum was tested for its bactericidal activity against strain W50 of P. gingivalis. The bacteria were grown to logarithmic phase in brain-heart infusion (BEI) broth. An aliquot of the bacterial culture was diluted to 5×10⁴ colony forming units (CFU) per ml in 10% bovine serum albumin in a balanced salt solution. The bactericidal assay reaction contained bacteria, polyclonal antiserum to inactivated PrtR-PrtK protein, a complement source consisting of normal human serum which was absorbed with protein G to remove antibodies, and the balanced salt solution. All reagents were added to the reaction to yield a 250 μl volume. Aliquots of 25 μl of the reaction were removed and plated in triplicate on BHI agar at times 0 and 60 minutes. The plates were incubated and colonies were counted the next day. The percent killing was calculated using the average of the three triplicate values at the 2 times. A representative example of data generated by the bactericidal assays is shown in Table 4. The results indicate that the polyclonal antiserum raised to the inactivated PrtR-PrtK is bactericidal for P. gingivalis. As illustrated by Table 4, controls show that the antiserum does not kill bacteria in the absence of complement, and that the complement source does not kill the bacteria in the absence of the antiserum, indicating that the bactericidal activity is antibody directed and complement mediated.

TABLE 4
Bactericidal activity of anti-(PrtR-PrtK) antibody

| | | | | | CFU at | CFU at | Percent |
|---|---|---|---|---|---|---|---|
| Sample | Antiserum | | Complement | | time 0 | time 60 | killing |
| 1 | 10 | μl | 22 | μl | 225 | 0 | 100% |
| 2 | 10 | μl | 0 | | 227 | 390 | 0% |
| 3 | 0 | | 22 | μl | 254 | 286 | 0% |

In further illustrating that the PrtR-PrtK protein possesses properties desirable of a vaccine antigen, pooled immune sera raised to strain W50 was shown to have cross-reactivity with heterologous strains. The pooled immune sera, prepared against PrtR-PrtK protein as described above, was examined for cross-reactivity with nine P. gingivalis strains from diverse clinical and geographical sources. Bacteria from each culture were harvested by swabs and suspended in PBS to an optical absorbance of 1.0 at 600 nm. A microliter of each suspension was applied to a nitrocellulose membrane and allowed to dry The membrane was incubated one hour at room temperature in a solution of 5% non-fat dry milk in PBS to block the residual binding sites of the membrane. The membrane was washed twice with PBS, and then immersed in the blocking solution containing the immune sera diluted to 1:1000. The membrane was incubated with the antibody overnight at 46° C. with gentle shaking. The membrane was washed three times with PBS and then incubated for 2 hours at room temperature with alkaline phosphatase conjugated recombinant protein G (1:1500 in PBS with 5% non-fat dry milk). The membrane was washed three times with PBS and bound antibody was detected by the addition of substrate. The immune sera reacted with all strains as strongly, or to a greater extent than, strain W50. Thus, the antibodies elicited by immunization of the PrtR-PrtK protein isolated from strain W50 cross-reacted with all heterologous strains tested.

For vaccine development, PrtR-PrtK may be purified from a host containing a recombinant vector which expresses PrtR-PrtK. Such hosts include, but are not limited to, bacterial transformants, yeast transformants, filamentous fungal transformants, and cultured cells that have been either infected or transfected with a vector which encodes PrtR-PrtK. Many methods are known for the introduction of a vaccine formulation into the human or animal to be vaccinated. These include, but are not limited to, intradermal, intramuscular, intraperitoneal, intravenous, subcutaneous, ocular, intranasal, and oral administration. The vaccine may further comprise a physiological carrier such as a solution, a polymer or liposomes; and an adjuvant, or a combination thereof.

### EXAMPLE 3

### Protective Efficacy of Immunisation With the PrtR-PrtK Complex in an Animal Model

Various preparations of purified P. gingivalis proteins were tested in the mouse abscess model. This model is loosely based on the methods described by Kesavalu et al (1992) [Infect Immun 60:1455-1464]. A typical experiment is outlined below. Briefly BALB/c mice were obtained from ARC (Perth, Australia) and were immunised subcutaneously in the scruff of the neck with the preparations and doses according to Table 5 before challenge with live P. gingivalis strain W50, which was given at 10 weeks of age. Mice were given 2 doses of vaccine at 4 and 1 weeks before challenge. Formalin killed P. gingivalis W50 cells were prepared by incubating an aliquot of cells in 0.5% (vol/vol) of buffered formal saline overnight at 4° C. The chloroform extract of P. gingivalis was prepared as detailed in Example 2. Purification of PrtR-PrtK complex was performed as detailed in Example 1. The PrtR-PrtK domains were prepared by taking the PrtR-PrtK complex and incubating in the presence of 50 mM 2-mercaptoethanol for 8 h at 4° C. This resulted in the breakdown of the PrtR-PrtK complex to domains that were 15-115 kDa proteins as shown by gel filtration FPLC and SDS-PAGE as performed in Example 1

All preparations were emulsified with an equal volume of Freund's Incomplete Adjuvant (FIA; Sigma) prior to injection.

Animals were bled before and 1 week after the immunisation schedule. Sera were screened by ELISA using a P. gingivalis sonicate (prepared as in Example 1) as the adsorbed antigen. The immunogenicity of the purified PrtR-PrtK complex is shown in FIG. 11

TABLE 5
Immunization schedule

| | No. of | | |
|---|---|---|---|
| Group | Doses | Treatment | n |
| 1 | 2 | 1 × 10⁹ Formalin killed P. gingivalis cells in FIA¹ | 11 |
| 2 | 2 | Chloroform extracted P. gingivalis | 10 |
| | | proteins in FIA | |
| 3 | 2 | Affinity purified P. gingivalis | 5 |
| | | PrtR-PrtK complex in FIA | |
| 4 | 2 | PrtR-PrtK Domains in FIA | 10 |
| 5 | 2 | Tris-cysteine buffer in FIA | 10 |
| 6 | 2 | Tris-cysteine buffer | 10 |

¹FIA = Freunds incomplete adjuvant

For the preparation of the bacterial challenge P. gingivalis cells were grown at 37° C. on lysed horse blood agar (HBA) plates until day 3 or 4 in an anaerobic chamber (Mark 3 Anaerobic Workstation, Don Whitley Scientific Limited; with an air mixture of 8% H₂, 12% CO₂, 80% N₂), then passaged into 20 ml of brain heart infusion broth (BHIB; Oxoid) supplemented with 0.5 g/L cysteine and 1 mg/L haemin for 24 hours in a standard incubator at 37° C. Finally, 3 ml of this culture was added to 400 ml of BHIB-cysteine media and incubated for approximately 15 hours in a standard incubator at 37° C., until the optical density at 650 nm reached 0.18. The cells were then pelleted by centrifigation at 10,000 g for 30 minutes using a JA10 rotor in a Beckman High Speed centrifuge and then resuspended to a final dilution of 3×10¹⁰ cells per ml in BHIB-cysteine media according to previously established growth curves for the W50 strain used in these experiments. Mice were marked for identification, their backs and chests shaved to make measurement of lesions possible, then weighed prior to inoculation with the challenge dose at a single site in the middle of the back. A 0.1 ml dose was given representing a predicted challenge dose of 3×10⁹ bacteria per mouse. The inoculum dose was confirmed by culturing various dilutions of the challenge dose on lysed HBA plates and examining the number of colonies 7 days later.

Following challenge mice were examined daily for the number and size of lesions on their body and their size estimated by measuring the approximate surface area in mm² involved. Previous experiments had shown that in unimmunized mice, lesions developed on the belly of the mice following inoculation of live bacteria into the back or side. Any distressed animals were culled. Observations were carried out over two weeks and a summary of one such experiment is summarised below in Table 6. In this experiment while a dose of 3×10⁹ bacteria per mouse was the desired number of bacteria, after plating out of the inoculum it was calculated that each mouse actually received a challenge dose of 3.17×10⁹ live P. gingivalis bacteria strain W50.

When mice were immunised with the various P. gingivalis fractions significant reductions (p<0.05) were seen in the size of the lesions with whole formalin killed P. gingivalis strain W50 cells (Group 1), the chloroform extracted proteins (Group 2) and the PrtR-PrtK complex (Group 3) when compared with the lesion size of the animals receiving FIA (Group 5) (Table 6). The PrtR-PrtK domains (Group 4) of the broken down PrtR-PrtK complex did not significantly reduce lesion size compared with the control (Group 5). These results clearly show that the complex works effectively as an immunogen whereas the PrtR-PrtK domains (15-115 kDa proteins) do not. The only group of animals that had a number of animals (40%) that exhibited no visible lesions at all was the PrtR-PrtK complex group (Group 3). All other groups, including formalin killed cells (Group 1), had all animals exhibiting visible lesions indicating that the PrtR-PrtK complex was a better immunogen than formalin killed cells.

TABLE 6
Immunisation with the PrtR-PrtK complex can
protect mice from challenge
with P. gingivalis

| | Lesion size | |
|---|---|---|
| Group | Mean maximum lesion size mm² | p* |
| 1 | 30.2 ± 28.4^{†} | 0.0008 |
| 2 | 39.0 ± 33.2 | 0.009 |
| 3 | 30.0 ± 36.0 | 0.0028 |
| 4 | 88.3 ± 32.2 | NS |
| 5 | 86.8 ± 41.1 | — |
| 6 | 201.7 ± 125.8 | 0.012 |

*probability calculated by Mann Whitney rank sum test comparing Group 5 with other groups
^{†}mean ± SD

### EXAMPLE 4

### Cloning and Sequence Analysis of the prtR and prtK Genes

### Bacterial Strains

P. gingivalis W50 was grown in modified BM medium supplemented with 1 μg/ml haemin in an atmosphere of 10% CO₂, 10% H₂ and 80% N₂ at 37° C. Escherichia coli JM109 and Escherichia coli LE392 were grown in LB medium at 37° C. Escherichia coli strains harbouring pUC18 plasmids were grown in LB medium supplemented with 100 μg/ml ampicillin at 37° C.

### Genomic Library Construction

Chromosomal DNA was isolated from P. gingivalis W50 as described by Smith et al., [Oral Microbiol. Immunol. 4:47-51 (1989)] except that cells were pelleted from a 500 ml late-exponential culture. The genomic library was constructed from BamHI partially-digested W50 DNA which was partially-filled with dGTP and dATP and ligated into LambdaGEM®-12 XhoI half-site arms (Promega) and packaged using Packagenee® (Promega).

prtR gene characterisation: The genomic library was screened using degenerate synthetic oligonucleotides derived from the N-terminal sequence information of the purified PrtR45. The oligonucleotide probes were based on the amino acid sequence YEGDIKD (antisense) and KDFVDWKNQ (sense) and were 5′ end-labelled using γ³²P ATP and T4 polynucleotide kinase. Approximately 1.5×10⁴ phage were screened by lifting onto Nylon membrane filters and hybridised with radiolabeled oligonucleotides overnight in hybridisation buffer: 6×SSC (SSC is 15 mM sodium citrate, 150 mM NaCl pH 8.0), 0.25% SDS, 5× Denhardt's solution and 100 μg/ml salmon sperm DNA at 44° C. Filters were washed extensively in a solution of 5×SSC containing 0.01% SDS (w/v) at 44° C. Positively-hybridising plaques were purified. Standard protocols for end-labelling of oligonucleotides and screening procedures were essentially as described in Sambrook et al. (1989) [Molecular Cloning: A Laboratory Manual, 2nd ed., Cold Spring Harbour Laboratory Press]. Lambda clone four with an insert size of approximately 15 kb was selected and this fragment contained the entire prtR gene. The 15 kb fragment was cut with appropriate restriction enzymes and the fragments generated subcloned into pUC18. Escherichia coli JM109 was transformed with the recombinant plasmids using electroporation.

prtK gene characterisation: The 5′ portion of the gene encoding PrtK was isolated from the same genomic library described above. The genomic library was screened using a degenerate synthetic oligonucleotide derived from the N-terminal sequence information of the purified PrtK48. The oligonucleotide probes were sense to the amino acid sequence DVYTDHGD and radiolabelled as described above Hybridisation and washing conditions were as described above except that the temperature was 48° C. and the filters were washed extensively in a solution of 3×SSC containing 0.01% SDS (w/v) at 48° C. Lambda clone 12 with an insert size of approximately 15 kb was selected and digested with BamHI and a 3.3 kb fragment was ligated into plasmid BamHI-BAP pUC18 and Escherichia coli JM109 transformed with the recombinant plasmid as described previously. Due to an internal BamHI site within prtK, the 3.3 kb BamHI fragment contained the 5′ portion of prtK which constituted the end of the lambda 12 clone. Sequence characterisation of the 3.3 kb BamHI fragment showed that the DNA sequence encoding PrtK48 contains an internal EcoRI site Subsequently, a second oligonucleotide probe (lysur) specific to the sequence THIGAH which is found within the PrtK48 was generated to determine a suitable strategy for cloning the 3′ end of prtK. Southern blot analysis of genomic DNA indicated that a 7.5 kb EcoRI fragment contained the entire 3′ portion of prtK. In order to characterise the 3′ end of the prtK gene a second genomic library was prepared. EcoRI digested DNA fragments of 6-8 kb were purified from an agarose gel and subsequently ligated to EcoRI digested Lambda Zap II-calf intestinal phosphatase-treated vector (Stratagene). The genomic library enriched for 6-8 kb P. gingivalis EcoRI fragments was packaged using GigapackIII Gold packaging extract (Stratagene) according to the manufacturer's instructions. The library was screened as described previously, using oligonucleotide lysur except that hybridisation temperatures were 42° C. and filters were washed to 3×SSC containing 0.01% SDS (w/v) at 42° C. In vivo excision of the Lambda Zap II positive genomic clone was performed (Stratagene instruction manual) to excise the pBluescript phagemid which was subsequently sequenced to generate the sequence information corresponding to the 3′end of the prtK gene.

DNA Sequencing Double-stranded plasmid template DNA prepared following the procedure of Li and Schweizer [Focus 15:19-20 (1993)] was sequenced in both directions using DNA sequence-derived, synthetic oligonucleotides, following the di-deoxy termination method [Proc. Natl. Acad. Sci. U.S.A. 74:5463-5467 (1977)], using the Sequenase version 2.0 nucleotide sequencing kit purchased from United States Biochemicals Nucleotide and protein sequence data were analysed using programme suites accessed by the Australian National Genomic Information Service (ANGIS).

### EXAMPLE 5

The following is an example of a proposed toothpaste formulation containing anti-(PrtR-PrtK) antibodies.

| Ingredient | % w/w |
|---|---|
| Dicalcium phosphate dihydrate | 50.0 |
| Glycerol | 20.0 |
| Sodium carboxymethyl cellulose | 1.0 |
| Sodium lauryl sulphate | 1.5 |
| Sodium lauroyl sarconisate | 0.5 |
| Flavour | 1.0 |
| Sodium saccharin | 0.1 |
| Chlorhexidine gluconate | 0.01 |
| Dextranase | 0.01 |
| Goat serum containing anti-(PrtR-PrtK) | 0.2 |
| Water | balance |

### EXAMPLE 6

The following is an example of a proposed toothpaste formulation.

| Ingredient | % w/w |
|---|---|
| Dicalcium phosphate dihydrate | 50.0 |
| Sorbitol | 10.0 |
| Glycerol | 10.0 |
| Sodium carboxymethyl cellulose | 1.0 |
| Sodium lauryl sulphate | 1.5 |
| Sodium lauroyl sarconisate | 0.5 |
| Flavour | 1.0 |
| Sodium saccharin | 0.1 |
| Sodium monofluorophosphate | 0.3 |
| Chlorhexidine gluconate | 0.01 |
| Dextranase | 0.01 |
| Bovine serum containing anti-(PrtR-PrtK) | 0.2 |
| Water | balance |

### EXAMPLE 7

The following is an example of a proposed toothpaste formulation.

| Ingredient | % w/w |
|---|---|
| Dicalcium phosphate dihydrate | 50.0 |
| Sorbitol | 10.0 |
| Glycerol | 10.0 |
| Sodium carboxymethyl cellulose | 1.0 |
| Lauroyl diethanolamide | 1.0 |
| Sucrose monolaurate | 2.0 |
| Flavour | 1.0 |
| Sodium saccharin | 0.1 |
| Sodium monofluorophosphate | 0.3 |
| Chlorhexidine gluconate | 0.01 |
| Dextranase | 0.01 |
| Bovine milk Ig containing anti-(PrtR-PrtK) | 0.1 |
| Water | balance |

### EXAMPLE 8

The following is an example of a proposed toothpaste formulation.

| Ingredient | % w/w |
|---|---|
| Sorbitol | 22.0 |
| Irish moss | 1.0 |
| Sodium Hydroxide (50%) | 1.0 |
| Gantrez | 19.0 |
| Water (deionised) | 2.69 |
| Sodium Monofluorophosphate | 0.76 |
| Sodium saccharine | 0.3 |
| Pyrophosphate | 2.0 |
| Hydrated alumina | 48.0 |
| Flavour oil | 0.95 |
| anti-(PrtR-PrtK) mouse monoclonal | 0.3 |
| sodium lauryl sulphate | 2.00 |

### EXAMPLE 9

The following is an example of a proposed liquid toothpaste formulation.

| Ingredient | % w/w |
|---|---|
| Sodium polyacrylate | 50.0 |
| Sorbitol | 10.0 |
| Glycerol | 20.0 |
| Flavour | 1.0 |
| Sodium saccharin | 0.1 |
| Sodium monofluorophosphate | 0.3 |
| Chlorhexidine gluconate | 0.01 |
| Ethanol | 3.0 |
| Equine Ig containing anti-(PrtR-PrtK) | 0.2 |
| Linolic acid | 0.05 |
| Water | balance |

### EXAMPLE 10

The following is an example of a proposed mouthwash formulation.

| Ingredient | % w/w |
|---|---|
| Ethanol | 20.0 |
| Flavour | 1.0 |
| Sodium saccharin | 0.1 |
| Sodium monofluorophosphate | 0.3 |
| Chlorhexidine gluconate | 0.01 |
| Lauroyl diethanolamide | 0.3 |
| Rabbit Ig containing anti-(PrtR-PrtK) | 0.2 |
| Water | balance |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A substantially purified antigenic complex for use in raising an antibody response directed against Porphyromonas gingivalis, the complex comprising at least one multimeric protein complex of arginine-specific and lysine-specific thiol endopeptidases each containing at least one adhesin domain, the complex having a molecular weight of greater than about 200 kDa.

2. A substantially purified antigenic complex as claimed in claim 1 in which the multimeric protein complex is associated with virulent strains of Porphyromonas gingivalis.

3. A substantially purified antigenic complex as claimed in claim 1 or claim 2 in which the multimeric protein complex has a molecular weight of about 294 to about 323 kDa.

4. A substantially purified antigenic complex as claimed in any one of claims 1 to 3 in which the multimeric protein complex is composed of 9 proteins.

5. A substantially purified antigenic complex as claimed in claim 4 in which the 9 proteins have the following N-terminal sequences:
DVYTDHGDLYNTPVRML,
YTPVEEKQNGRMIVIVAKKYEGD,
SGQAEIVLEAHDVWNDGSGYQILLDADHDQYGQVIPSDTHFL,
PQSVWIERTVDLPAGTKYVAFR,
ANEAKVVLAADNVWGDNTGYQFLLDA,
AINEAKVVLAADNVWGDNTGYQFLLDA,
PQSVWIIERTVDLPAGTKYVAFR,
ADFTETFESSTHGEAPAEWTTIDA,
ADFTETFESSTHGEAPAEWTTIDA.

6. A substantially purified antigenic complex as claimed in claim 5 in which the 9 proteins are PrtK48, PrtR45, PrtR44, PrtK39, PrtK44, PrtR27, PrtR17, PrtK15 and PrtR15.

7. A substantially purified antigenic complex as claimed in claim 1 in which the thiol endopeptidases are rendered inactive.

8. A substantially purified antigenic complex as claimed in claim 7 in which the thiol endopeptidases are rendered inactive by oxidation.

9. A substantially purified antigenic complex as claimed in claim 7 in which the thiol endopeptidases are rendered inactive by mutation.

10. A substantially purified antigenic complex as claimed in claim 1 in which the multimeric protein complex is encoded by the DNA sequence shown in FIGS. 8B and 9B.

11. A composition for use in eliciting an immune response directed against Porphyromonas gingivalis, the composition comprising an effective amount of the complex as claimed in any one of claims 1 to 10 and a suitable adjuvant and/or acceptable carrier.

12. An antibody preparation comprising antibodies specifically directed against the complex as claimed in any one of claims 1 to 10.

13. An antibody preparation as claimed in claim 12 in which the antibodies are polyclonal antibodies.

14. An antibody preparation as claimed in claim 12 in which the antibodies are monoclonal antibodies.

15. A method of treating a subject suffering from Porphyromonas gingivalis infection, the method comprising administering to the subject an amount of the antibody preparation as claimed in any one of claims 12 to 14 effective to at least partially neutralize the PrtR-PrtK complex of Porphyromonas gingivalis.

16. A method as claimed in claim 15 in which the antibody preparation is administered as a mouth wash or as a dentifrice.

17. A method of reducing the prospect of P. gingivalis infection in an individual and/or severity of disease, the method comprising administering to the individual an amount of the composition as claimed in claim 11 effective to induce an immune response in the individual directed against P. gingivalis.

18. A recombinant host cell, the host cell being transformed with a DNA sequence(s) encoding PrtR-PrtK operatively linked to control sequences such that under appropriate conditions the host cell expresses PrtR-PrtK.

19. A recombinant host cell as claimed in claim 18 in which the host cell is an oral commensal.

20. A recombinant host cell as claimed in claim 18 or claim 19 in which the host cell is transformed with the DNA sequences shown in FIG. 8b and FIG. 9b.
